(19) United States
(12) Patent Application Publication
(10) Pub. No.: US 2020/0030262 A1
(43) Pub. Date: Jan. 30, 2020

(54) THERAPEUTIC SIGMA 1 RECEPTOR AGONISTS FOR TREATING LONG QT SYNDROME TYPE 1, TYPE 2, TYPE 6, TYPE 8 AND RELATED CHANNELOPATHIES

(71) Applicant: The Trustees of Columbia University in the City of New York, New York, NY (US)

(72) Inventors: Masayuki Yazawa, New York, NY (US); LouJin Song, Cambridge, MA (US); Kumi Morikawa, Shiga (JP); Jose Quejada, New York, NY (US); Ramsey Bekdash, Cabin John, MD (US)

(21) Appl. No.: 16/593,296

(22) Filed: Oct. 4, 2019

Related U.S. Application Data
(63) Continuation-in-part of application No. PCT/US2018/025821, filed on Apr. 3, 2018.
(60) Provisional application No. 62/481,266, filed on Apr. 4, 2017.

(51) Int. Cl. A61K 31/15; A61K 31/5375; A61K 31/485; A61P 9/06

(52) U.S. Cl. CPC A61K 31/15; A61P 9/06; A61K 31/485; A61K 31/5375

(57) ABSTRACT

The present invention relates to compounds and methods for increasing or agonizing Sigma 1 Receptors and its pathway for treating long QT syndrome (LQTS), and in particular Timothy Syndrome (TS), LQT8, LQT1, LQT2, and LQT6. Additionally, the invention relates to small molecule based therapies and combinations for treating Timothy Syndrome (TS), LQT8, LQT1, LQT2, LQT6 and related channelopathies.

### CROSS-REFERENCE TO RELATED APPLICATIONS

The present application is a continuation-in-part of International Patent Application Ser. No. PCT/US2018/025821, filed Apr. 3, 2018, which claims priority to U.S. Provisional Patent Application Ser. No. 62/481,266 filed Apr. 4, 2017, which is incorporated herein by reference in its entirety.

### GOVERNMENT SUPPORT

This invention was made with government support under HL131087, HL138486 and HL142239, all awarded by National Institutes of Health. The government has certain rights in the invention.

### SEQUENCE LISTING

The instant application contains a Sequence Listing which is being filed electronically in ASCII format and is hereby incorporated by reference in its entirety. Said ASCII copy, created on Mar. 28, 2018, is named 01001-005273-WO0_ST25.txt and is 1,104 bytes in size.

### FIELD OF THE INVENTION

The present invention relates to compounds and methods for increasing or agonizing Sigma 1 Receptors and its pathway for treating long QT syndrome (LQTS), and in particular Timothy Syndrome (TS), LQT8, LQT1, LQT2 and LQT6. Additionally, the invention relates to small molecule based therapies and combinations for treating Timothy Syndrome (TS), LQT8, LQT1, LQT2, LQT6 and related channelopathies.

### BACKGROUND

Despite a substantial reduction in age-adjusted rates of death from cardiovascular causes during the past 40 to 50 years, cardiovascular disease remains the most common cause of natural death in developed countries. Sudden death due to cardiac arrhythmia is estimated to account for approximately 50 percent of all deaths from cardiovascular causes (Huikuri H V, et al. (2001)). In specific, the risk of sudden death due to genetic and drug-induced prolongation of QT interval (long QT syndrome, LQTS) is a major concern for patients, clinicians and pharmaceutical companies. Genetic LQTS has an estimated prevalence of 1 in 7,000 individuals and it results from mutations in at least 13 genes that encode cardiac ion channel genes or other regulatory molecules (Crotti L, et al. (2013); Mahida S, et al. (2013); Venetucci L, et al. (2012). Manifestations of LQTS during fetal or neonatal life usually indicate a severe form of the disease. Drug-induced LQTS is a side effect of many approved drugs and is a common cause of drug failure in clinical trials (Mahida S, et al. (2013), Paakkari I. (2002)). Despite our knowledge of many of the genes that cause LQTS, the mechanisms that underlie LQTS in humans are incompletely understood. Animal models of human LQTS using rodents have proved to be problematic because the mouse resting heart rate is approximately 10 fold faster than that of humans. Mouse cardiomyocytes have different electrical properties from their human counterparts. Previous experiments using rodent models and clinical trials could not predict potential side effects; for example, cisapride had been approved by US FDA as a gastroprokinetc agent. However, it was withdrawn from US market in 2000 because approximately 80 people died due to its side effect that causes QT prolongation, resulting in lethal arrhythmia and ventricular tachycardia (Paakkari I. (2002)). Therefore, better models including human cell culture systems are needed to investigate the molecular mechanisms of human cardiac diseases and to identify new therapeutics in this field.

Timothy syndrome (TS, Long QT Syndrome Type 8, LQT8) is an autosomal dominant disorder characterized by multisystem dysfunctions including lethal arrhythmia, congenital heart defects and autism (Splawski I, et al. (2004)). The disease is caused by one gain-of-function mutation in the CACNA1C gene encoding L-type voltage-gated calcium channel Cav1.2, and the mutation usually leads to ineffective channel inactivation. There are currently very few options for therapeutic treatment of patients with TS and none of the currently used drugs are very effective. To date, several attempts have been made to develop new therapeutics for treating TS and related conditions. However, they have exhibited limitations including undesired side effects and/or toxicities. Therefore, there is a need to develop new effective therapeutics for TS. In addition to the Timothy syndrome mutation, other mutations in CACNA1C have been reported to cause LQT8 (Fukuyama, et al. 2014). Among them, the A582D mutation leads to an ineffective channel inactivation, similar to the Timothy syndrome mutation although to a less extent. Patients with the A582D mutation in CACNA1C develop severe cardiac arrhythmia and the mutation is inheritable. Currently there is no effective therapeutic interventions that can be used to manage the cardiac arrhythmia in LQT8 patients caused by mutations other than the Timothy syndrome mutation and new therapeutics are needed for treating LQT8.

Long QT syndrome Type 1(LQT1) is a genetic disorder caused by mutations in the KCNQ1 gene encoding voltage-gated potassium channel subunit Kv7.1. The disease is characterized by a prolongation of the QT interval on ECG and the occurrence of life-threatening cardiac events, commonly triggered by adrenergic stimulation (Wu, et al. 2016). The estimated LQT1 disease prevalence is approximately 1:7500 in the general population, which contributes to about ⅓ of the total patient population with LQTS (Giudicessi, et al. 2013). Although several therapeutic options are available to treat LQT1, the disease is still not well managed in some patients due to heterogeneous clinical manifestations of the disease and not fully characterized disease mechanisms. Therefore new therapeutics will be useful for treating LQT1 and potentially to be used in combination with the current available therapeutics to achieve a better management of the disease conditions in LQT1 patients.

Long QT syndrome Type 2 (LQT2) is another major type in genetic cardiac arrhythmias with prolonged QT interval. It is caused by mutations such as A561V in the hERG channels encoded by KCNH2 gene. LQT1 and LQT2 account for 75-80% of cases with long QT syndrome (Mahida, et al. 2013). There are no available specific therapeutics for patients with LQT2. Therefore, there is an unmet need for treatment and prevention of LQT2.

Long QT syndrome Type 6 (LQT6) is a subtype of LQTS that is caused by mutations in the KCNE2 gene encoding the potassium channel beta subunit MiRP1. MiRP1 has been showed to modulate pacemaker current and is associated with sinus bradycardia (Nawathe, et al. 2013). It has also been showed to modulate HERG potassium channel activity and induce pro-arrhythmic effects (Lu, et al. 2003). So far there is no effective therapeutics for LQT6 patients and new therapeutics are needed to treat the disease.

### SUMMARY OF THE INVENTION

In certain embodiments, the present invention relates to a method for increasing sigma-1 receptor activity in a subject in need thereof, comprising administering to the subject an effective amount of fluvoxamine or PRE-084 or dextromethorphan, or derivatives thereof, combinations thereof, or a pharmaceutically acceptable salt thereof.

In additional embodiments, the subject exhibits one or more symptoms associated with Timothy Syndrome (TS), LQT8, LQT1, LQT2, LQT6 or a related channelopathy.

In yet additional embodiments, one or more symptoms exhibit improvement and comprise any one or combination of improvements selected from the group consisting of increasing the spontaneous beating rate, decreasing the contraction irregularity, enhancing the voltage-dependent inactivation of Cav1.2 channels, rescuing the abnormal spontaneous and paced action potentials; and alleviating the abnormal spontaneous calcium transients in affected or diseased cardiomyocytes.

In additional embodiments, the method further comprises administering an effective amount of Myoseverin-B and/or PHA-793887, and/or Roscovitine and/or CR8 and/or DRF053, or derivatives thereof.

In additional embodiments, the invention relates to a method for treating Timothy Syndrome (TS), LQT8, LQT1, LQT2, LQT6 or related channelopathy in a subject in need thereof comprising increasing or agonizing sigma-1 receptor activity in the subject in an amount to alleviate at least one symptom associated with TS, LQT8, LQT1, LQT2, LQT6 or related channelopathy.

In certain embodiments, increasing the activity is by gene therapy.

In certain embodiments, the increasing or agonizing is by administering an effective amount of fluvoxamine or PRE-084 or dextromethorphan, or derivatives thereof, combinations thereof, or a pharmaceutically acceptable salt thereof.

In certain embodiments, one or more symptoms exhibiting improvement comprise any one or combination of improvements selected from the group consisting of increasing the spontaneous beating rate, decreasing the contraction irregularity, enhancing the voltage-dependent inactivation of Cav1.2 channels, rescuing the abnormal spontaneous and paced action potentials; and alleviating the abnormal spontaneous calcium transients in affected or diseased cardiomyocytes.

In additional embodiments, the method further comprises administering an effective amount of Myoseverin-B and/or PHA-793887, or derivatives thereof, and/or Roscovitine and/or CR8 and/or DRF053.

In yet additional embodiments, the invention relates to methods for treating or reducing risk of a cardiac arrhythmia in a subject in need thereof, comprising administering to the subject a therapeutically effective amount of fluvoxamine or PRE-084 or dextromethorphan, or derivatives thereof, combinations thereof, or a pharmaceutically acceptable salt thereof.

In further embodiments, the subject is a mammal. In yet further embodiments, the mammal is a human.

### BRIEF DESCRIPTION OF THE DRAWINGS

For the purpose of illustrating the invention, there are depicted in drawings certain embodiments of the invention. However, the invention is not limited to the precise arrangements and instrumentalities of the embodiments depicted in the drawings.

The patent or application file contains at least one drawing executed in color. Copies of this patent or patent application publication with color drawing(s) will be provided by the Office upon request and payment of the necessary fee.

FIGS. 1A-J Fluvoxamine alleviated the phenotypes in Timothy syndrome cardiomyocytes. FIG. 1A-B show the analysis of the contraction irregularity and the beating rate of Timothy syndrome cardiomyocytes before and 2 hours after 5 μM Fluvoxamine (Fluvo) treatment (n=16 from two Timothy syndrome lines derived from two TS patients. ***P<0.005, ****P<0.001; Student's t-test, paired; Data are mean±s.e.m. The data was normalized to the corresponding control group for each treatment group for the presentation in this figure and the statistical test was conducted using the raw data. Fluvo, Fluvoxamine). FIG. 1C shows representative recordings of Ba²⁺ currents in the Fluvoxamine (Fluvo, 5 μM, 2 hr treatment) treated and untreated Timothy syndrome cardiomyocyte. FIG. 1D shows voltage-dependent inactivation of Cav1.2 in the Fluvoxamine (Fluvo, 5 μM, 2 hr) treated and untreated Timothy syndrome cardiomyocytes (n=5 per group from two Timothy syndrome lines. **P<0.01; Student's t-test; Data are mean±s.e.m.). FIG. 1E shows the current-voltage relationship of the Timothy syndrome cardiomyocytes with and without Fluvoxamine treatment (5 μM, 2 hr, n=5 per group from two Timothy syndrome lines. Data are mean±s.e.m.). FIG. 1F shows representative spontaneous calcium transient traces from single Timothy syndrome cardiomyocyte before and 30 minutes after the treatment of 5 μM Fluvoxamine. Fluvoxamine treatment (5 μM, 30 min) alleviated the abnormal spontaneous calcium transients in Timothy syndrome cardiomyocytes. FIGS. 1G-H are graphs showing quantification of spontaneous calcium transient duration and half decay time in Timothy syndrome cardiomyocytes before and after the treatment of 5 μM Fluvoxamine for 30 minutes. Fluvoxamine treatment (5 μM, 30 min) significantly reduced the spontaneous calcium transient duration and half decay time in Timothy syndrome cardiomyocytes (n=52 from two Timothy syndrome lines. *P<0.05; Student's t-test, paired; Data are mean±s.e.m.). FIGS. 1I-J show the beneficial effects of Fluvoxamine (Fluvo, 5 μM, 2 hr) were blocked by a co-treatment of a sigma 1 receptor antagonist NE-100 (5 μM, 2 hr). The other serotonin reuptake transporter inhibitor (SSRI) paroxetine with a low affinity for Sigma 1 receptor did not have any effects on Timothy syndrome cardiomyocytes (n=10 from one Timothy syndrome line. **P<0.01, ****P<0.001; Student's t-test, paired; Data are mean±s.e.m.).

FIGS. 2A-O PRE-084 alleviated the phenotypes in Timothy syndrome cardiomyocytes. FIGS. 2A-B are graphs showing the analysis of the contraction irregularity and the beating rate of Timothy syndrome cardiomyocytes before and 2 hours after 5 μM PRE-084 treatment (n=10 from one Timothy syndrome line. *P<0.05, ****P<0.001; Student's t-test, paired; Data are mean±s.e.m.). FIG. 2C shows representative recordings of Ba²⁺ currents in the PRE-084 (PRE, 5 μM, 2 hr treatment) treated and untreated Timothy syndrome cardiomyocytes. FIG. 2D is a graph showing voltage-dependent inactivation of Cav1.2 in the PRE-084 (PRE-084, 5 μM, 2 hr) treated and untreated Timothy syndrome cardiomyocytes (n=4 for the control group without treatment, and n=5 for the group with PRE-084 treatment, from two Timothy syndrome lines. **P<0.01; Student's t-test; Data are mean±s.e.m.). FIG. 2E shows representative recordings of Ba²⁺ currents in the PRE-084 (PRE, 5 μM, <1 min, acute treatment) treated and untreated Timothy syndrome cardiomyocyte. FIG. 2F is a graph showing voltage-dependent inactivation of Cav1.2 in the acute PRE-084 (PRE, 5 μM) treated and untreated Timothy syndrome cardiomyocytes (acute treatment of PRE-084, <1 min; n=4 per group from two Timothy syndrome lines. n.s., not significant; Student's t-test; Data are mean±s.e.m.). FIG. 2G are traces showing representative spontaneous action potential recordings from Timothy syndrome cardiomyocyte with and without PRE-084 treatment (5 μM, 2 hr). Dashed line indicates 0 mV. FIG. 2H is a graph showing quantification of spontaneous action potential durations (APD50 and APD90) from Timothy syndrome cardiomyocytes with and without PRE-084 treatment (5 μM, 2 hr) (n=4 for the control group without treatment, and n=5 for the group with PRE-084 treatment, from two Timothy syndrome lines. **P<0.01; Student's t-test; Data are mean±s.e.m.). FIG. 2I are traces showing representative paced (0.2 Hz) action potential recordings from Timothy syndrome cardiomyocyte without any treatment, with Fluvoxamine treatment (Fluvo, 5 μM, 2 hr) or with PRE-084 treatment (PRE, 5 μM, 2 hr). FIG. 2J is a graph showing quantification of paced action potential duration (APD90) from Timothy syndrome cardiomyocytes without any treatment, with Fluvoxamine treatment (Fluvo, 5 μM, 2 hr) or with PRE-084 treatment (PRE, 5 μM, 2 hr) (n=9 for the control group without treatment, n=9 the group with Fluvoxamine treatment, and n=13 for the group with PRE-084 treatment, from two Timothy syndrome lines. **P<0.01; One-way ANOVA with Bonferroni post-hoc; Data are mean±s.d.). FIG. 2K are a representative spontaneous calcium transient trace from single Timothy syndrome cardiomyocyte before treatment and another trace 30 minutes after the treatment of 5 μM PRE-084. PRE-084 treatment (5 μM, 30 min) alleviated the abnormal spontaneous calcium transients in Timothy syndrome cardiomyocytes. FIGS. 2L-M are graphs showing the quantification of spontaneous calcium transient duration and half decay time in Timothy syndrome cardiomyocytes before and after the treatment of 5 μM PRE-084 for 30 minutes. PRE-084 treatment (5 μM, 30 min) significantly reduced the spontaneous calcium transient duration and half decay time in Timothy syndrome cardiomyocytes (n=44 from two Timothy syndrome lines. **P<0.01; Student's t-test, paired; Data are mean±s.e.m.). FIGS. 2N-O are graphs showing the analysis of the contraction irregularity and the beating rate of Timothy syndrome cardiomyocytes before and 7 days after 10 μM PRE-084 or Fluvoxamine (Fluvo) treatment (n=8 from one Timothy syndrome line. **P<0.01, ***P<0.005, ****P<0.001; Student's t-test, paired; Data are mean±s.e.m.). The control group without treatment completely quitted contraction and exhibited observable signs of cell death at day 27 while the PRE-084 and Fluvoxamine treated groups showed an enhanced rhythmic contraction and cell survival.

FIGS. 3A-H are graphs and traces showing the effects of PRE-084 or Fluvoxamine on healthy control cardiomyocytes. FIG. 3A-B are graphs showing the analysis of the contraction irregularity and the beating rate of control cardiomyocytes before and 2 hours after 5 μM Fluvoxamine (Fluvo) or PRE-084 treatment (n=25 from three control lines. ****P<0.001; Student's t-test, paired; Data are mean±s.e.m.). FIG. 3C is a representative spontaneous calcium transient traces from single control cardiomyocyte before and 30 minutes after the treatment of 5 μM Fluvoxamine. Fluvoxamine treatment (5 μM, 30 min) did not have significant effects on the calcium transients in control cardiomyocytes. FIGS. 3D-E are graphs showing quantification of spontaneous calcium transient duration and half decay time in control cardiomyocytes before and after the treatment of 5 μM Fluvoxamine for 30 minutes. Fluvoxamine treatment (5 μM, 30 min) didn't have significant effects on the spontaneous calcium transient duration and half decay time in control cardiomyocytes (n=35 from three control lines. n.s., not significant; Student's t-test, paired; Data are mean±s.e.m.). FIG. 3F are representative spontaneous calcium transient traces from single control cardiomyocyte before and 30 minutes after the treatment of 5 μM PRE-084. PRE-084 treatment (5 μM, 30 min) did not have significant effects on the calcium transients in control cardiomyocytes. FIGS. 3G-H are graphs showing quantification of spontaneous calcium transient duration and half decay time in control cardiomyocytes before and after the treatment of 5 μM PRE-084 for 30 minutes. PRE-084 treatment (5 μM, 30 min) did not have significant effects on the spontaneous calcium transient duration and half decay time in control cardiomyocytes (n=36 from three control lines. n.s., not significant; Student's t-test, paired; Data are mean±s.e.m.).

FIGS. 4A-K are graphs and blots showing likely mechanisms underlying the beneficial effects of Fluvoxamine and PRE-084 on Timothy syndrome cardiomyocytes. FIG. 4A is a graph showing the effects of the pre-treatment of PRE-084 and Fluvoxamine (Fluvo) on endogenous cdk5 activity in Timothy syndrome cardiomyocytes. The cells were pre-treated with 5 or 10 μM PRE-084 or 10 μM Fluvoxamine before being collected for cdk5 immunoprecipitation and cdk5 activity assay. PHA-793887(PHA) was added to the reaction mix before the cdk5 activity assay as a positive control (n=7 from 10 μM Fluvoxamine group and n=8 for all the other groups from one Timothy syndrome line. *P<0.05, **P<0.01; One-way ANOVA with Dunnett's post-hoc; Data are mean±s.e.m.). FIGS. 4B-C are graphs showing the gene expression of cdk5 normalized to GAPDH before and after the treatment of 5 μM Fluvoxamine or PRE-084 (n=4 per group from one Timothy syndrome line. n.s., not significant; One-way ANOVA with Dunnett's post-hoc; Data are mean±s.e.m.). FIG. 4D shows representative blots of p35 protein expression before and after the treatment of 5 μM Fluvoxamine (Fluvo) or PRE-084. FIG. 4E is a graph showing quantification of p35 protein expression before and two hours after the treatment of 5 μM Fluvoxamine (Fluvo) or PRE-084 (n=14 for TS control and n=9 for Fluvo or PRE-084 treated group from one Timothy syndrome line. n.s., not significant; One-way ANOVA with Dunnett's post-hoc; Data are mean±s.e.m.). FIG. 4F is a blot showing Fluvoxamine (Fluvo, 10 μM, 7-day treatment) decreased the expression of p35 in Timothy syndrome cardiomyocytes. FIG. 4G shows representative images of cdk5 and p35 protein expression before and after the treatment of 5 μM PRE-084 for two hours. FIG. 4H is a graph showing quantification of cdk5 protein expression before and two hours after the treatment of 5 μM PRE-084 for two hours (n=5 per group from two Timothy syndrome lines. *P<0.05; Student's t-test; Data are mean±s.e.m.). FIG. 4I are representative images of cdk5 and sigma 1 receptor co-immunoprecipitation (Co-IP). The cell lysates from control and Timothy syndrome cardiomyocytes were incubated with CDK5 antibody-conjugated beads for CDK5 IP and then used for western blotting. Cdk5 co-IPed with Sigma 1 receptor in both control and Timothy syndrome cardiomyocytes. FIGS. 4J-K are graphs showing the analysis of the contraction irregularity and the beating rate of Timothy syndrome cardiomyocytes before and 2 hours after 1 μM PRE-084 treatment or 1 μM Myoseverin-B (Myo-B, Roscovitine analog, not a potent cdk5 inhibitor) treatment or 1 μM PHA-793887 (PHA, cdk5 inhibitor) treatment or a combination of PRE-084 and either Myoseverin-B or PHA-793887 or both (n=9 for luM PRE group and n=10 for all the other groups from two iPSC lines derived from two TS patients. *P<0.05, **P<0.01, ***P<0.005, ****P<0.001; Student's t-test, paired; Data are mean±s.e.m. The data was normalized to the corresponding control group for each treatment group for the presentation in this figure and the statistical test was conducted using the raw data.).

FIG. 5 is a diagram showing the related potential mechanism that the sigma-1 receptor agonists may act through to alleviate the phenotypes in Timothy syndrome.

FIGS. 6A-J are graphs and traces showing the effects of Fluvoxamine on LQT8, LQT1 and LQT6 cardiomyocytes. FIG. 6A-B are a graph and images showing the pedigree of the family with inherited LQT8 caused by the A582D mutation and the iPSCs derived from the two patients. FIG. 6C shows representative recordings of Ba²⁺ currents in the control cardiomyocytes and LQTS8 cardiomyocyte. FIG. 6D shows voltage-dependent inactivation (VDI %) of Cav1.2 in the control cardiomyocytes and LQTS8 cardiomyocytes. (n=5 for control group from two lines and n=4 for LQTS8 group from one line. **P<0.01; Student's t-test; Data are mean±s.e.m.). FIG. 6E shows representative recordings of Ba²⁺ currents in the Fluvoxamine (Fluvo, 5 μM, 2 hr treatment) treated and untreated LQTS8 cardiomyocyte. FIG. 6F shows voltage-dependent inactivation of Cav1.2 in the Fluvoxamine (Fluvo, 5 μM, 2 hr) treated and untreated LQTS8 cardiomyocytes (n=11 per group from one line. **P<0.01; Student's t-test; Data are mean±s.d.). FIG. 6G are traces showing representative paced (0.5 Hz) action potential recordings from LQTS1 cardiomyocyte without any treatment and with Fluvoxamine treatment (Fluvo, 5 μM, 2 hr). FIG. 6H is a graph showing quantification of paced action potential duration (APD90) from LQTS1 cardiomyocytes without any treatment and with Fluvoxamine treatment (Fluvo, 5 μM, 2 hr) (n=5 for the control group without treatment and n=9 for the group with Fluvo treatment, from one line. *P<0.05; Student's t-test; Data are mean±s.d.). FIG. 6I are traces showing representative paced (0.5 Hz) action potential recordings from LQTS6 cardiomyocyte without any treatment and with Fluvoxamine treatment (Fluvo, 5 μM, 2 hr). FIG. 6J is a graph showing quantification of paced action potential duration (APD90) from LQTS6 cardiomyocytes without any treatment and with Fluvoxamine treatment (Fluvo, 5 μM, 2 hr) (n=4 for the control group without treatment and n=9 for the group with Fluvo treatment, from one line. **P<0.01; Student's t-test; Data are mean±s.d.).

FIGS. 7A-D show the effect of dextromethorphan on the phenotypes in cardiac action potentials in Timothy syndrome iPSC-derived cardiomyocytes. FIG. 7A shows representative traces of paced action potentials in Timothy syndrome iPSC-derived cardiomyocytes untreated or treated with dextromethorphan (Dxm, 5 uM, 2 hr). FIG. 7B is graph of the action potential duration (APD90, 90% reduction from peak) in Timothy syndrome iPSC-derived cardiomyocytes untreated or treated with dextromethorphan (Dxm, 5 uM, 2 hr) showing that the action potential duration was significantly shortened by dextromethorphan. FIG. 7C are representative traces of paced action potentials in isogenic control (Ctrl) iPSC-derived cardiomyocytes untreated or treated with dextromethorphan (5 uM, 2 hr). FIG. 7D is a graph of action potential duration (APD90, 90% reduction from peak) in isogenic control (Ctrl) iPSC-derived cardiomyocytes untreated or treated with dextromethorphan (5 uM, 2 hr) showing the action potential duration was not changed by dextromethorphan in the control cells. Student t-test was used (*P<0.05; **P<0.01; n.d., no difference).

FIGS. 8A-I show testing of dextromethorphan on cardiac calcium handling and ion channels in human iPSC model of Timothy syndrome. FIG. 8A show representative traces of time-course GCaMP6f-based calcium imaging in Timothy syndrome iPSC-derived cardiomyocytes treated with dextromethorphan (Dxm, 5 uM, 2 hr). FIG. 8B is a graph of cardiac transient frequency in Timothy syndrome iPSC-derived cardiomyocytes treated with dextromethorphan (Dxm, 5 uM, 2 hr), showing that cardiac transient frequency was significantly increased by dextromethorphan in Timothy syndrome iPSC-derived cardiomyocytes. FIG. 8C is a graph of cardiac transient duration in Timothy syndrome iPSC-derived cardiomyocytes treated with dextromethorphan (Dxm, 5 uM, 2 hr), showing that cardiac transient duration was significantly shortened by dextromethorphan in Timothy syndrome iPSC-derived cardiomyocytes. FIG. 8D are representative traces of Ba²⁺ current in Timothy syndrome iPSC-derived cardiomyocytes untreated or treated with dextromethorphan (5 uM, 2 hr). FIG. 8E shows a graph of voltage-dependent calcium channel inactivation in Timothy syndrome iPSC-derived cardiomyocytes untreated or treated with dextromethorphan, showing that voltage-dependent calcium channel inactivation was significantly increased by dextromethorphan in Timothy syndrome iPSC-derived cardiomyocytes. FIG. 8F are representative traces of Ca²⁺ current in Timothy syndrome iPSC-derived cardiomyocytes untreated or treated with dextromethorphan (5 uM, 2 hr). FIG. 8G is a graph of calcium channel inactivation in Timothy syndrome iPSC-derived cardiomyocytes untreated or treated with dextromethorphan, showing that calcium channel inactivation was significantly increased by dextromethorphan in Timothy syndrome iPSC-derived cardiomyocytes. FIG. 8H shows representative traces of I_{Kr} current in Timothy syndrome iPSC-derived cardiomyocytes untreated (n=5) or treated with dextromethorphan (5 uM, 2 hr, n=7). E-4031-sensitive currents were measured and analyzed as I_{Kr} current. FIG. 8I is a graph of I_{Kr} current in Timothy syndrome iPSC-derived cardiomyocytes untreated or treated with dextromethorphan, showing that I_{Kr} current was significantly increased by dextromethorphan in Timothy syndrome iPSC-derived cardiomyocytes. Student t-test was used (*P<0.05; **P<0.01).

FIGS. 9A-D shows the effect of dextromethorphan on the phenotypes in ion channels in long QT syndrome type 2. FIG. 9A are representative traces of action potentials in long QT syndrome type 2 iPSC-derived cardiomyocytes treated with dextromethorphan (Dxm, 5 uM, 2 hr). FIG. 9B is a graph of action potential duration in long QT syndrome type 2 iPSC-derived cardiomyocytes untreated or treated with dextromethorphan, showing action potential duration was significantly shortened by dextromethorphan in long QT syndrome type 2 iPSC-derived cardiomyocytes. FIG. 9C shows representative traces of I_{Kr} current in long QT syndrome type 2 iPSC-derived cardiomyocytes untreated (n=6) or treated with dextromethorphan (5 uM, 2 hr, n=7). E-4031-sensitive currents were measured and analyzed as I_{Kr} current. FIG. 9D is a graph of I_{Kr} current long QT syndrome type 2 iPSC-derived cardiomyocytes untreated or treated with dextromethorphan, showing that I_{Kr} current was significantly increased by dextromethorphan. Student t-test was used (*P<0.05; **P<0.01).

FIGS. 10A-D shows the effect of dextromethorphan on the phenotypes in QT interval of Timothy syndrome mouse model. FIG. 10A shows the targeting design of Timothy syndrome mutant (G406R in the calcium channel Cacna1c gene) in mouse Rosa26 locus. FIG. 10B shows the experimental design for inducing the expression of Timothy syndrome mutant with Dxm treatment (approximately 28 mg/kg). Dxm concentration in the plasma was characterized using normal mice (see FIG. 11A). FIG. 10C are representative traces of electrocardiography (ECG) in Timothy syndrome mice and the control littermate (3 months old) treated with dextromethorphan (in water, free drinking) or without treatment at day 4. FIG. 10D is a graph of the QT intervals in the indicated mice showing that the QT intervals were significantly shortened by dextromethorphan in Timothy syndrome mouse line. One-way ANOVA with multiple comparisons were used (**P<0.01; n.d., no difference).

FIGS. 11A-C shows the further characterization of dextromethorphan effects on Timothy syndrome mouse model. FIG. 11A is a summary of Dxm measurement in the plasma in normal mice (n=5). Dxm was provided into water in day 1 at 8 am. FIG. 11B are the detailed analysis procedures in ECG traces in Timothy syndrome mice treated with dextromethorphan or without treatment at day 4 (used in FIG. 10C). FIG. 11C is a graph of QT intervals in control mice and untreated and treated Timothy Syndrome mice showing that QT intervals were significantly shortened by dextromethorphan in Timothy syndrome mouse line at day 11. One-way ANOVA with multiple comparisons were used (**P<0.01; n.d., no difference).

FIG. 12 is a graph showing dextromethorphan tested on long QT syndrome type 1 -action potential duration long QT syndrome type 1 iPSC-derived cardiomyocytes untreated and treated with dextromethorphan, showing that action potential duration was significantly shortened by dextromethorphan in long QT syndrome type 1 iPSC-derived cardiomyocytes. Student t-test was used (**P<0.01).

### DETAILED DESCRIPTION

Aspects of the present invention relate in part to the molecular mechanism of inhibiting CDK5 and the beneficial effects of inhibiting CDK5 on Timothy syndrome cardiomyocytes. The present results provide new insights into the regulation of cardiac calcium channels and the development of novel therapeutics for Timothy syndrome patients.

In certain embodiments, the invention relates to administering an effective amount of a compound such as a sigma-1 receptor agonist including fluvoxamine or PRE-084 or dextromethorphan, or derivatives thereof, or a combination thereof, to a patient with TS, LQT8, LQT1, LQT2, LQT6 or a related channelopathy. In additional embodiments, the sigma-1 receptor agonist (or combination thereof) is administered in combination with at least one additional therapeutic agent, such as a CDK5 inhibitor or a p35 inhibitor, combinations thereof, but not limited to these agents.

### Definitions

Terms have the meanings ascribed to them in the text unless expressly stated to the contrary. It must be noted that, as used herein, the singular forms “a”, “an,” and “the” include plural references unless the context clearly dictates otherwise. In addition, the following terms have the following meanings.

The term “effective amount” of a compound is a quantity sufficient to achieve a desired therapeutic and/or prophylactic effect, for example, an amount which results in the alleviation, prevention of, or a decrease in the symptoms associated with a disease that is being treated, e.g., Long QT syndrome (LQTS), or in particular Timothy Syndrome (TS), LQT8, LQT1, LQT2 or LQT6.

“Activation,” “stimulation,” and “treatment,” as it applies to cells or to receptors, may have the same meaning, e.g., activation, stimulation, or treatment of a cell or receptor with a ligand, unless indicated otherwise by the context or explicitly. “Ligand” encompasses natural and synthetic ligands, e.g., cytokines, cytokine variants, analogues, muteins, and binding compounds derived from antibodies. “Ligand” also encompasses small molecules, e.g., peptide mimetics of cytokines and peptide mimetics of antibodies. “Activation” can refer to cell activation as regulated by internal mechanisms as well as by external or environmental factors. “Response,” e.g., of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming.

“Activity” of a molecule may describe or refer to the binding of the molecule to a ligand or to a receptor, to catalytic activity; to the ability to stimulate gene expression or cell signaling, differentiation, or maturation; to antigenic activity, to the modulation of activities of other molecules, and the like. “Activity” of a molecule may also refer to activity in modulating or maintaining cell-to-cell interactions, e.g., adhesion, or activity in maintaining a structure of a cell, e.g., cell membranes or cytoskeleton. “Activity” can also mean specific activity, e.g., [catalytic activity]/[mg protein], or [immunological activity]/[mg protein], concentration in a biological compartment, or the like. “Activity” may refer to modulation of components of the innate or the adaptive immune systems.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. “Administration” and “treatment” can refer, e.g., to therapeutic, pharmacokinetic, diagnostic, research, and experimental methods. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

“Treat” or “treating” means to administer a therapeutic agent, such as a composition containing any compound or therapeutic agent of the present invention, internally or externally to a subject or patient having one or more disease symptoms, or being suspected of having a disease or being at elevated at risk of acquiring a disease, for which the agent has therapeutic activity. Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom (also referred to as the “therapeutically effective amount”) may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom. While an embodiment of the present invention (e.g., a treatment method or article of manufacture) may not be effective in alleviating the target disease symptom(s) in every subject, it should alleviate the target disease symptom(s) in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treatment,” as it applies to a human, veterinary, or research subject, refers to therapeutic treatment, prophylactic or preventative measures, to research and diagnostic applications. “Treatment” as it applies to a human, veterinary, or research subject, or cell, tissue, or organ, encompasses contact of a cdk5 inhibitor to a human or animal subject, a cell, tissue, physiological compartment, or physiological fluid.

### Pharmaceutical Compositions and Administration

Compounds described herein, and their salts and solvates, and physiologically functional derivatives thereof, may be employed alone or in combination with other therapeutic agents for the treatment of conditions as described herein. Combination therapies according to the present invention thus comprise the administration of at least one compound as described herein, or a pharmaceutically acceptable salt or solvate thereof, or a physiologically functional derivative thereof, and the use of at least one other pharmaceutically active agent. The compound(s) of compound/s described herein and the other pharmaceutically active agent(s) may be administered together or separately and, when administered separately this may occur simultaneously or sequentially in any order. The amounts of the compound(s) as described herein, and the other pharmaceutically active agent(s) and the relative timings of administration will be selected in order to achieve the desired combined therapeutic effect.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimize the activity and/or stability and/or physical characteristics, such as solubility, of the therapeutic ingredient. It will be clear also that, where appropriate, the therapeutic ingredients may be used in optically pure form.

The combinations referred to above may conveniently be presented for use in the form of a pharmaceutical composition and thus pharmaceutical compositions comprising a combination as defined above together with a pharmaceutically acceptable diluent or carrier represent a further aspect of the invention.

The individual compounds of such combinations may be administered either sequentially or simultaneously in separate or combined pharmaceutical compositions. Preferably, the individual compounds will be administered simultaneously in a combined pharmaceutical composition. Appropriate doses of known therapeutic agents will be readily appreciated by those skilled in the art.

To prepare pharmaceutical or sterile compositions of the present invention, the compound is admixed with a pharmaceutically acceptable carrier or excipient. See, e.g., Remington's Pharmaceutical Sciences and U.S. Pharmacopeia: National Formulary, Mack Publishing Company, Easton, Pa. (1984).

In certain embodiments, an effective amount of fluvoxamine, PRE-084, or dextromethorphan, or derivatives thereof, or combinations thereof, is administered to a patient in need thereof. In certain embodiments, an effective amount of dextromethorphan ranges from about 5 to about 10 ng/ml.

Formulations of therapeutic and diagnostic agents may be prepared by mixing with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N.Y.). Additional agents, such as polysorbate 20 or polysorbate 80, may be added to enhance stability.

Toxicity and therapeutic efficacy of the compositions, administered alone or in combination with another agent, can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration. In an embodiment of the invention, a composition of the invention is administered to a subject in accordance with the Physicians' Desk Reference 2003 (Thomson Healthcare; 57th edition (Nov. 1, 2002)).

The mode of administration can vary. Suitable routes of administration include oral, rectal, transmucosal, intestinal, parenteral; intramuscular, subcutaneous, intradermal, intramedullary, intrathecal, direct intraventricular, intravenous, intraperitoneal, intranasal, intraocular, inhalation, insufflation, topical, cutaneous, transdermal, or intra-arterial.

In particular embodiments, the compound or agents can be administered by an invasive route such as by injection (see above). In further embodiments of the invention, the compound, or pharmaceutical composition thereof, is administered intravenously, subcutaneously, intramuscularly, intraarterially, intra-articularly (e.g. in arthritis joints), intratumorally, or by inhalation, aerosol delivery. Administration by non-invasive routes (e.g., orally; for example, in a pill, capsule or tablet) is also within the scope of the present invention.

In yet another embodiment, the compound such as a sigma-1 receptor agonist such as fluvoxamine or PRE-084 or dextromethorphan, or derivatives thereof, or combinations thereof, is administered in combination with at least one additional therapeutic agent, such as a CDK5 inhibitor or a p35 inhibitor, combinations thereof, but not limited to these agents.

“Homology” refers to sequence similarity between two polynucleotide sequences or between two polypeptide sequences when they are optimally aligned. When a position in both of the two compared sequences is occupied by the same base or amino acid monomer subunit, e.g., if a position in each of two DNA molecules is occupied by adenine, then the molecules are homologous at that position. The percent of homology is the number of homologous positions shared by the two sequences divided by the total number of positions compared x100. For example, if 6 of 10 of the positions in two sequences are matched or homologous when the sequences are optimally aligned then the two sequences are 60% homologous. Generally, the comparison is made when two sequences are aligned to give maximum percent homology.

“Isolated nucleic acid molecule” means a DNA or RNA of genomic, mRNA, cDNA, or synthetic origin or some combination thereof which is not associated with all or a portion of a polynucleotide in which the isolated polynucleotide is found in nature, or is linked to a polynucleotide to which it is not linked in nature. For purposes of this disclosure, it should be understood that “a nucleic acid molecule comprising” a particular nucleotide sequence does not encompass intact chromosomes. Isolated nucleic acid molecules “comprising” specified nucleic acid sequences may include, in addition to the specified sequences, coding sequences for up to ten or even up to twenty or more other proteins or portions or fragments thereof, or may include operably linked regulatory sequences that control expression of the coding region of the recited nucleic acid sequences, and/or may include vector sequences.

The phrase “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to use promoters, polyadenylation signals, and enhancers.

A nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. As used herein, the expressions “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, “polymerase chain reaction” or “PCR” refers to a procedure or technique in which specific nucleic acid sequences, RNA and/or DNA, are amplified as described in, e.g., U.S. Pat. No. 4,683,195. Generally, sequence information from the ends of the region of interest or beyond is used to design oligonucleotide primers. These primers will be identical or similar in sequence to opposite strands of the template to be amplified. The 5′ terminal nucleotides of the two primers can coincide with the ends of the amplified material. PCR can be used to amplify specific RNA sequences, specific DNA sequences from total genomic DNA, and cDNA transcribed from total cellular RNA, bacteriophage or plasmid sequences, etc. See generally Mullis et al. (1987) Cold Spring Harbor Symp. Quant. Biol. 51:263; Erlich, ed., (1989) PCR TECHNOLOGY (Stockton Press, N.Y.) As used herein, PCR is considered to be one, but not the only, example of a nucleic acid polymerase reaction method for amplifying a nucleic acid test sample comprising the use of a known nucleic acid as a primer and a nucleic acid polymerase to amplify or generate a specific piece of nucleic acid.

“Inhibitors” and “antagonists,” or “activators” and “agonists,” refer to inhibitory or activating molecules, respectively, e.g., for the activation of, e.g., a ligand, receptor, cofactor, a gene, cell, tissue, or organ. A modulator of, e.g., a gene, a receptor, a ligand, or a cell, is a molecule that alters an activity of the gene, receptor, ligand, or cell, where activity can be activated, inhibited, or altered in its regulatory properties. The modulator may act alone, or it may use a cofactor, e.g., a protein, metal ion, or small molecule. Inhibitors are compounds that decrease, block, prevent, delay activation, inactivate, desensitize, or down regulate, e.g., a gene, protein, ligand, receptor, or cell. Activators are compounds that increase, activate, facilitate, enhance activation, sensitize, or up regulate, e.g., a gene, protein, ligand, receptor, or cell. An inhibitor may also be defined as a compound that reduces, blocks, or inactivates a constitutive activity. An “agonist” is a compound that interacts with a target to cause or promote an increase in the activation of the target. An “antagonist” is a compound that opposes the actions of an agonist. An antagonist prevents, reduces, inhibits, or neutralizes the activity of an agonist. An antagonist can also prevent, inhibit, or reduce constitutive activity of a target, e.g., a target receptor, even where there is no identified agonist.

To examine the extent of inhibition, for example, samples or assays comprising a given, e.g., protein, gene, cell, or organism, are treated with a potential activator or inhibitor and are compared to control samples without the inhibitor. Control samples, i.e., samples not treated with antagonist, are assigned a relative activity value of 100%. Inhibition is achieved when the activity value relative to the control is about 90% or less, typically 85% or less, more typically 80% or less, most typically 75% or less, generally 70% or less, more generally 65% or less, most generally 60% or less, typically 55% or less, usually 50% or less, more usually 45% or less, most usually 40% or less, preferably 35% or less, more preferably 30% or less, still more preferably 25% or less, and most preferably less than 25%. Activation is achieved when the activity value relative to the control is about 110%, generally at least 120%, more generally at least 140%, more generally at least 160%, often at least 180%, more often at least 2-fold, most often at least 2.5-fold, usually at least 5-fold, more usually at least 10-fold, preferably at least 20-fold, more preferably at least 40-fold, and most preferably over 40-fold higher.

Endpoints in activation or inhibition can be monitored as follows. Activation, inhibition, and response to treatment, e.g., of a cell, physiological fluid, tissue, organ, and animal or human subject, can be monitored by an endpoint. The endpoint may comprise a predetermined quantity or percentage of, e.g., indicia of inflammation, oncogenicity, or cell degranulation or secretion, such as the release of a cytokine, toxic oxygen, or a protease. The endpoint may comprise, e.g., a predetermined quantity of ion flux or transport; cell migration; cell adhesion; cell proliferation; potential for metastasis; cell differentiation; and change in phenotype, e.g., change in expression of gene relating to inflammation, apoptosis, transformation, cell cycle, or metastasis (see, e.g., Knight (2000) Ann. Clin. Lab. Sci. 30:145-158; Hood and Cheresh (2002) Nature Rev. Cancer 2:91-100; Timme, et al. (2003) Curr. Drug Targets 4:251-261; Robbins and Itzkowitz (2002) Med. Clin. North Am. 86:1467-1495; Grady and Markowitz (2002) Annu. Rev. Genomics Hum. Genet. 3:101-128; Bauer, et al. (2001) Glia 36:235-243; Stanimirovic and Satoh (2000) Brain Pathol. 10:113-126).

An endpoint of inhibition is generally 75% of the control or less, preferably 50% of the control or less, more preferably 25% of the control or less, and most preferably 10% of the control or less. Generally, an endpoint of activation is at least 150% the control, preferably at least two times the control, more preferably at least four times the control, and most preferably at least ten times the control. The endpoints of activation using Fluvoxamine or PRE-084 on TS cardiomyocytes is to restore the voltage-dependent inactivation percentage (defined by the current intensity at 350 ms from the peak in the voltage-clamp recordings to record Ba²⁺ currents) value of Cav1.2 in TS cardiomyocytes to the level of control cardiomyocytes (approximately 60%).

“Small molecule” is defined as a molecule with a molecular weight that is less than 10 kDa, typically less than 2 kDa, preferably less than 1 kDa, and most preferably less than about 500 Da. Small molecules include, but are not limited to, inorganic molecules, organic molecules, organic molecules containing an inorganic component, molecules comprising a radioactive atom, synthetic molecules, peptide mimetics, and antibody mimetics. As a therapeutic, a small molecule may be more permeable to cells, less susceptible to degradation, and less apt to elicit an immune response than large molecules. Small molecules, such as peptide mimetics of antibodies and cytokines, as well as small molecule toxins, have been described (see, e.g., Casset, et al. (2003) Biochem. Biophys. Res. Commun. 307:198-205; Muyldermans (2001) J. Biotechnol. 74:277-302; Li (2000) Nat. Biotechnol. 18:1251-1256; Apostolopoulos, et al. (2002) Curr. Med. Chem. 9:411-420; Monfardini, et al. (2002) Curr. Pharm. Des. 8:2185-2199; Domingues, et al. (1999) Nat. Struct. Biol. 6:652-656; Sato and Sone (2003) Biochem. J. 371:603-608; U.S. Pat. No. 6,326,482 issued to Stewart, et al).

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

### EXAMPLES

### Example 1—Fluvoxamine and PRE-084 for Treating LQTS Including Timothy Syndrome

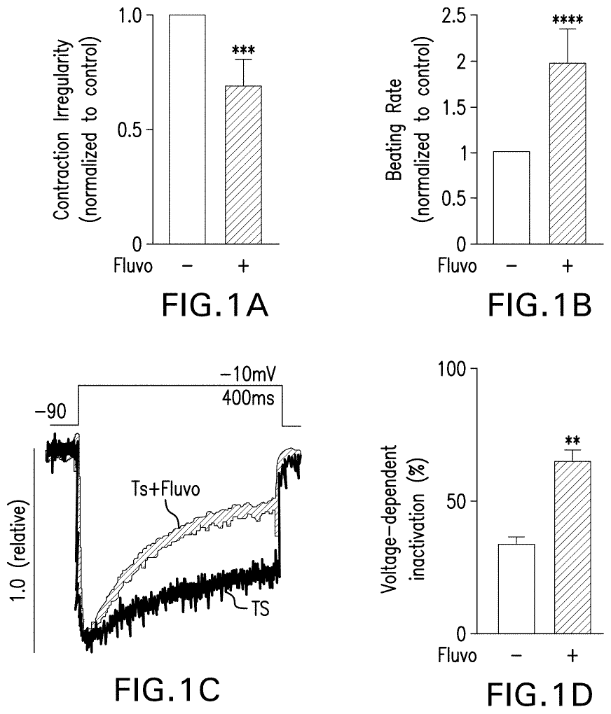

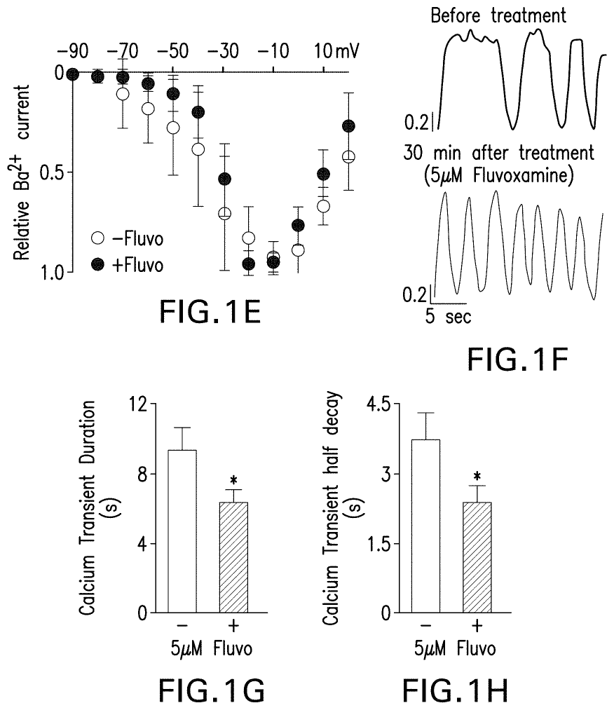

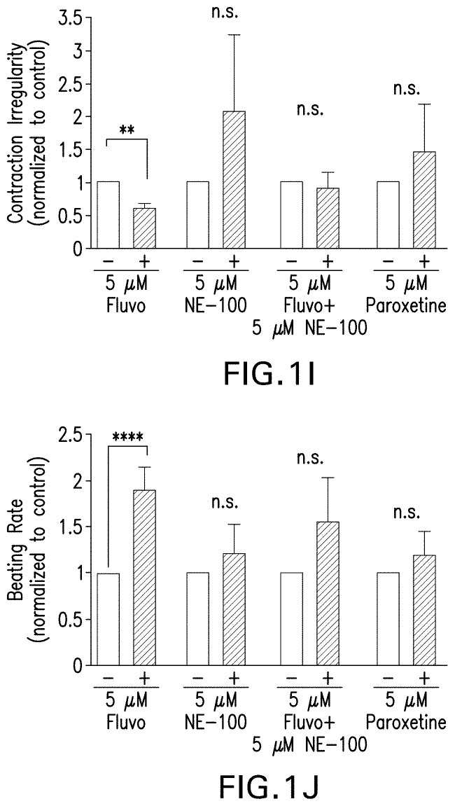

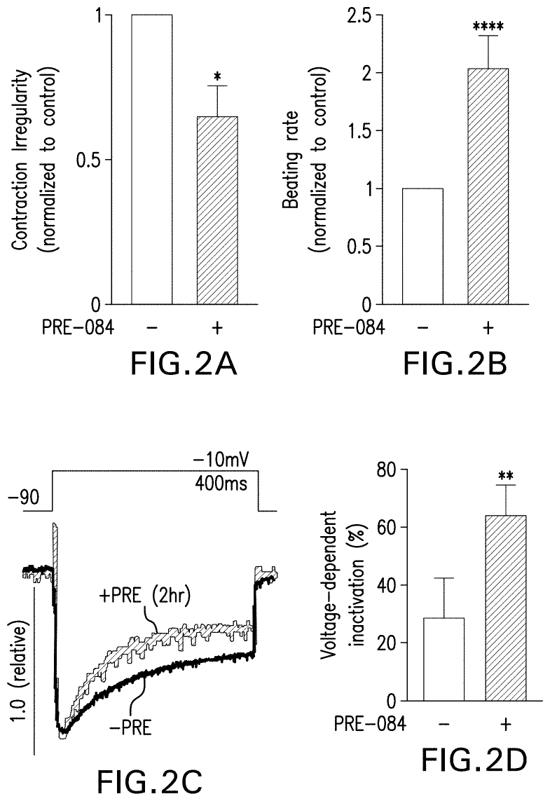

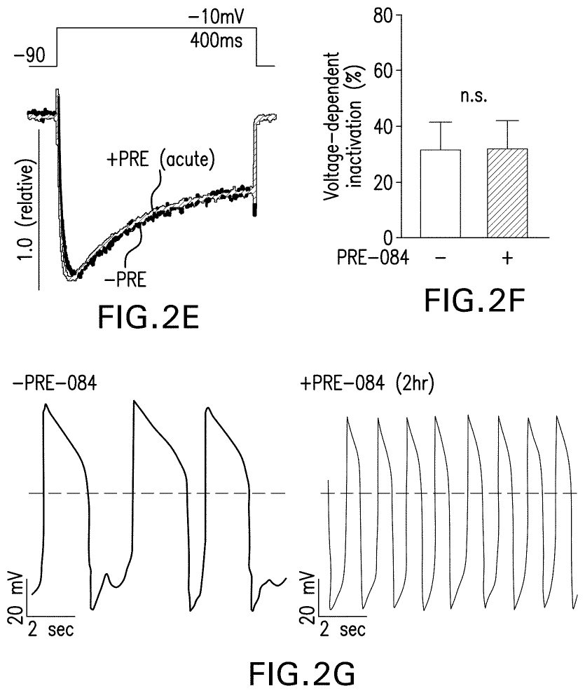

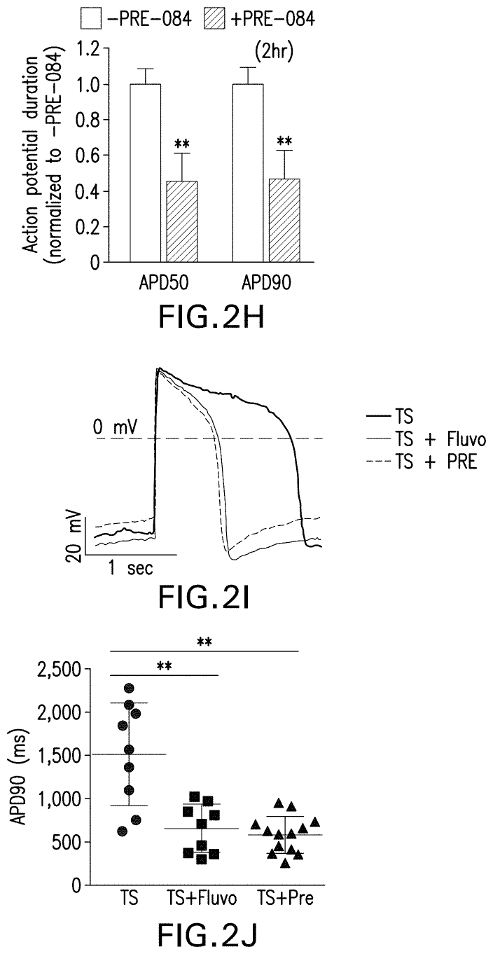

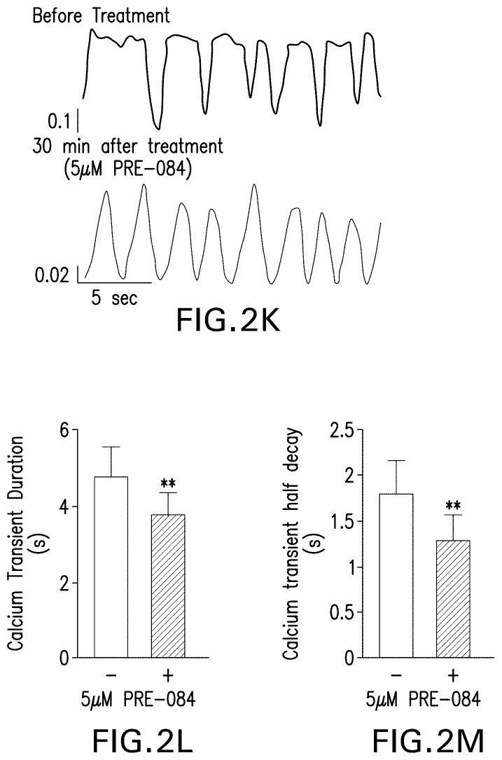

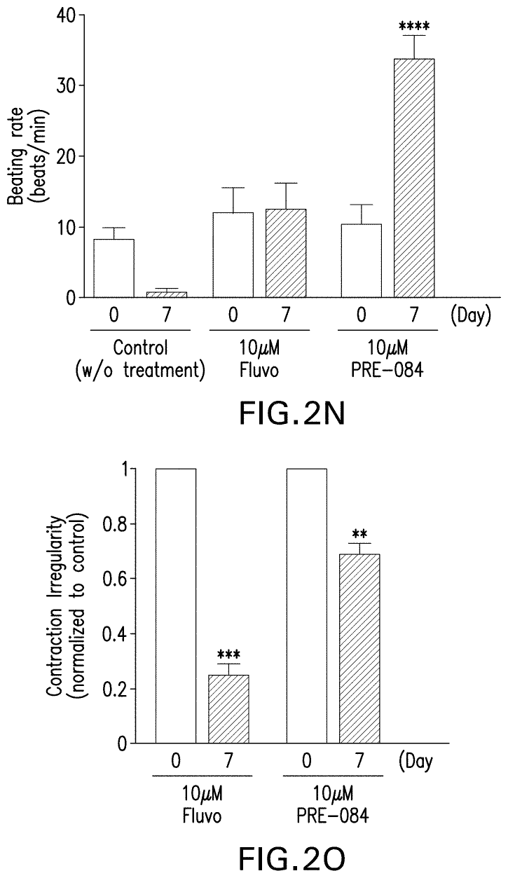

It has been reported that sigma 1 receptor is likely to be involved in the protein degradation of p35 in neurons (Tsai et al., 2015). For the present experiments, an FDA approved SSRI, Fluvoxamine, which is a sigma 1 receptor agonist was chosen as a potential therapeutic to test on the Timothy syndrome cardiomyocytes. The results suggest that 5 μM Fluvoxamine treatment significantly decreased the contraction irregularity, increased the spontaneous beating rate, enhanced the voltage-dependent inactivation of Cav1.2 channels, reduced the paced action potential duration and alleviated the abnormal spontaneous calcium transients, which are all features exhibited in Timothy syndrome cardiomyocytes (FIGS. 1A-1H and FIGS. 2I-2J). These results indicate that Fluvoxamine is beneficial for Timothy syndrome cardiomyocytes, rescuing many of the TS phenotypes. Since Fluvoxamine is not only a sigma 1 receptor agonist but also an SSRI, NE-100 (a potent sigma 1 receptor antagonist) and paroxetine (an SSRI with low affinity for sigma 1 receptor and the same affinity as Fluvoxamine for serotonin reuptake transporter) were tested to validate the sigma 1 receptor as the primary target of Fluvoxamine. The results suggest that the beneficial effects of Fluvoxamine can be blocked by a co-treatment with NE-100, and paroxetine has no beneficial effects on Timothy syndrome cardiomyocytes (FIGS. 1I and 1J), validating sigma 1 receptor as the primary target that accounts for the beneficial effects of Fluvoxamine.

Two other potent and selective sigma 1 receptor agonists were selected for testing on Timothy syndrome cardiomyocytes: PRE-084 and SA4503. The compound SA4503 stopped the contractions of Timothy syndrome cardiomyocytes at the dose between 1 μM to 5 μM. When the dose of SA4503 was lowered to 0.1 μM, the inhibitory effects of this compound on contraction was alleviated but there was no obvious improvement in the beating rate or contraction irregularity of Timothy syndrome cardiomyocytes with SA4503 treatments. This could be due to the potential inhibitory effect of SA4503 compound on sodium channels (Balasuriya et al., 2012; Gao et al., 2012; Johannessen et al., 2009). Derivatives of SA4503 that only activates Sigma 1 receptor without inhibiting sodium channels could also be therapeutics for Timothy syndrome.

The compound PRE-084 has been reported to have little effects on sodium channels (Johannessen et al., 2009). 5 μM PRE-084 treatment significantly increased the spontaneous beating rate, decreased the contraction irregularity, enhanced the voltage-dependent inactivation of Cav1.2 channels, rescued the abnormal spontaneous and paced action potentials and alleviated the abnormal spontaneous calcium transients in Timothy syndrome cardiomyocytes (FIGS. 2A-2M). The effects of long-term repeated treatments of Fluvoxamine and PRE-084 on Timothy syndrome cardiomyocytes were also analyzed. The cardiomyocytes were treated with Fluvoxamine or PRE-084 twice a day at the dose of 10 μM for seven days. The seven-day treatment of 10 μM Fluvoxamine or PRE-084 significantly enhanced the spontaneous beating rate and reduced the contraction irregularity in the Timothy syndrome cardiomyocytes (FIGS. 2N-2O), in contrast to the fact that the majority of the Timothy syndrome cardiomyocytes without any treatment quitted contractions at the end of the treatment period. The results suggest that long-term repeated treatment of Fluvoxamine or PRE-084 could improve the functions of the Timothy syndrome cardiomyocytes more significantly.

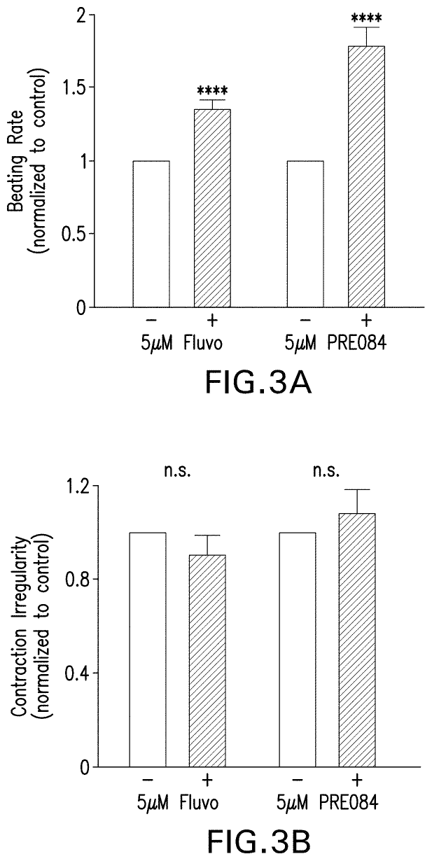

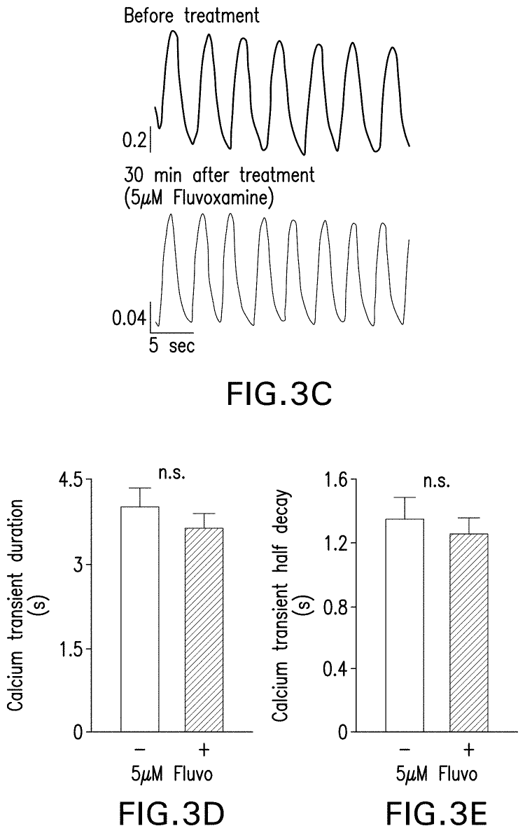

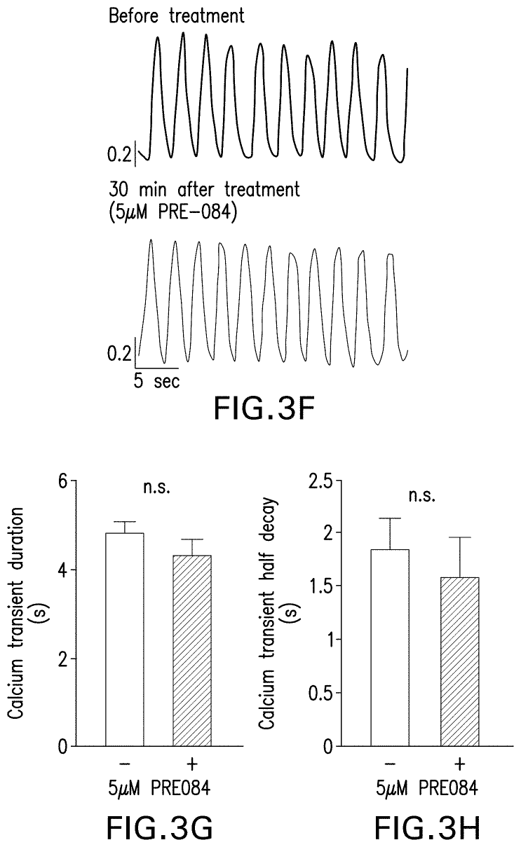

In addition, the effects of Fluvoxamine and PRE-084 were examined on the functions of healthy control cardiomyocytes. The effects of these two compounds on the contraction of control cardiomyocytes was evaluated. The results suggest that the treatment of 5 μM Fluvoxamine or PRE-084 for 2 hours significantly enhanced the spontaneous beating rate of control cardiomyocytes but did not significantly affect the contraction rhythmicity of the control cardiomyocytes (FIGS. 3A-3B). Next, the effects of Fluvoxamine and PRE-084 on the spontaneous normal calcium transients in control cardiomyocytes was evaluated. The treatment of 5 μM Fluvoxamine or PRE-084 did not significantly affect the normal calcium transients, calcium transient duration or half decay time in control cardiomyocytes (FIGS. 3C-3H). Overall, the results indicate that there are no significant side effects of Fluvoxamine or PRE-084 treatment on the contractions and the calcium transients in control cardiomyocytes.

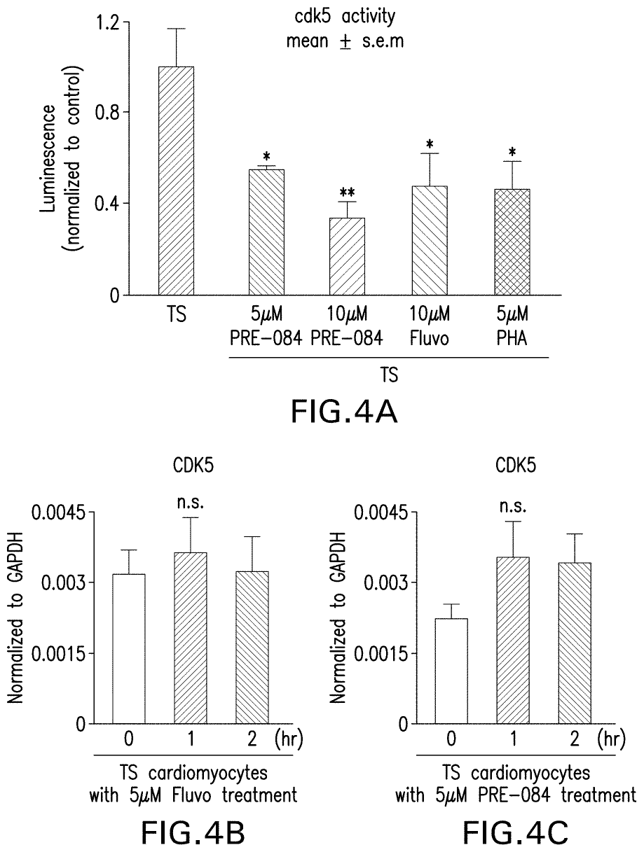

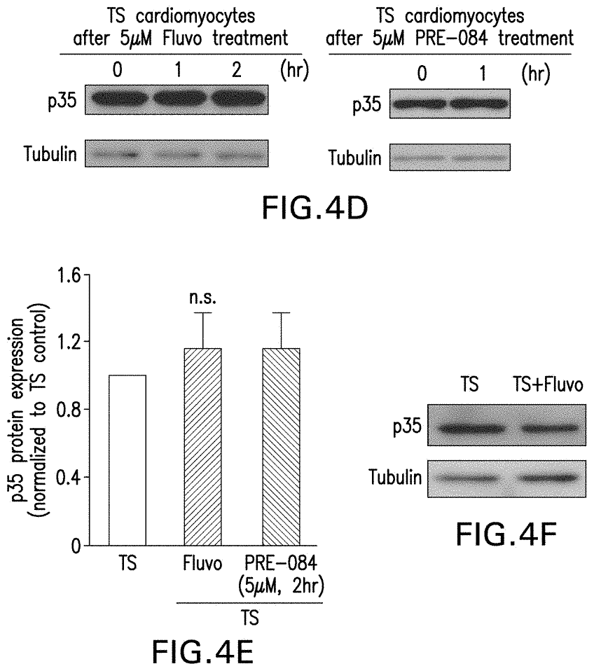

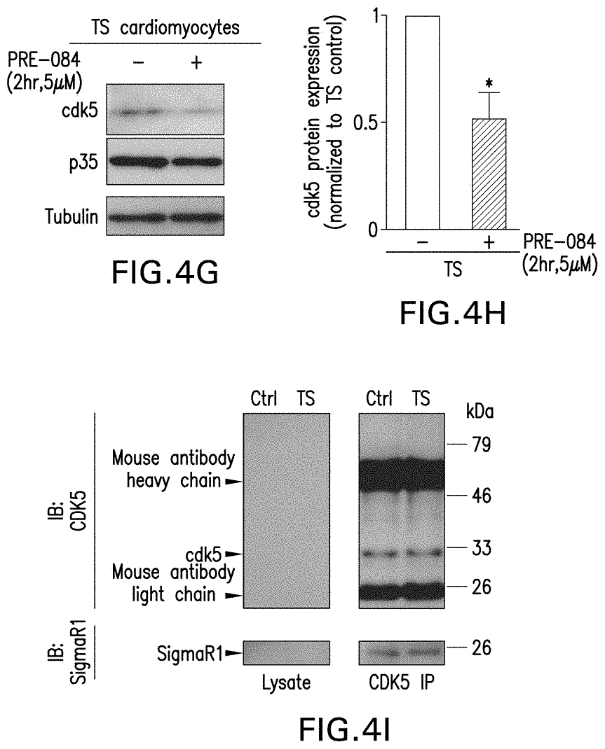

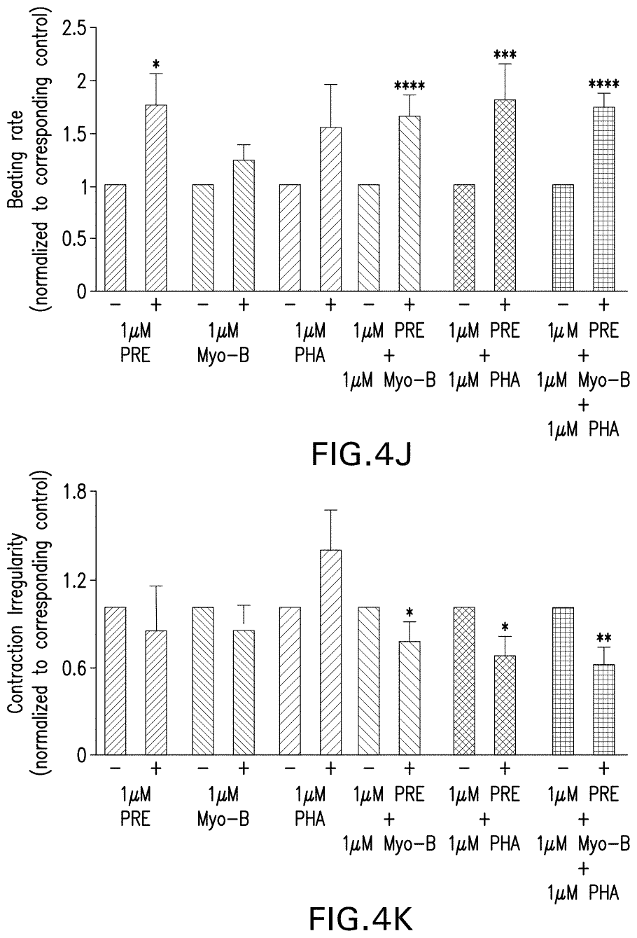

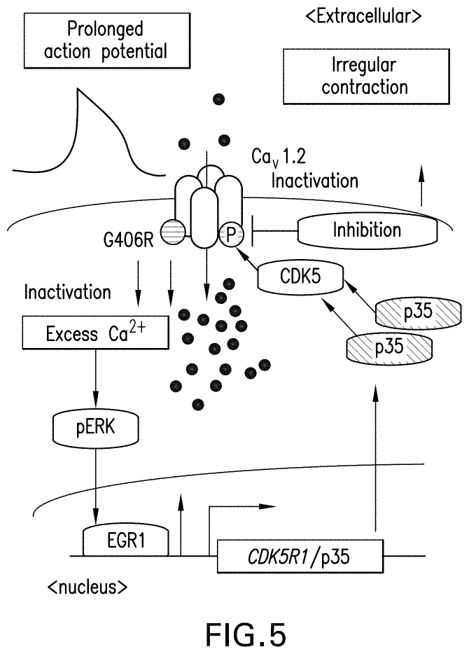

Moreover, to explore the mechanisms underlying the beneficial effects of Fluvoxamine and PRE-084, the effects of Fluvoxamine or PRE-084 were examined on the endogenous cdk5 activity in Timothy syndrome cardiomyocytes. These results suggest that the pre-treatment of 5 μM or 10 μM PRE-084, or 10 μM Fluvoxamine for two hours significantly decreased cdk5 activity in Timothy syndrome cardiomyocytes (FIG. 4A). The pre-treatment of the compounds refers to the procedure that the cardiomyocytes were treated with the compound in culture media for two hours before being collected and used for cdk5 activity assay. To investigate how Fluvoxamine or PRE-084 treatment could affect cdk5 activity, the gene expression of cdk5 before and after the treatment of Fluvoxamine or PRE-084 was examined. No significant change in the gene expression of cdk5 before and two hours after the treatment of Fluvoxamine or PRE-084 in Timothy syndrome cardiomyocytes (FIGS. 4B-4C) was observed. The protein expression of cdk5 activator p35 before and after the treatment of Fluvoxamine or PRE-084 was also analyzed. The results suggest that there was not significant change in p35 protein expression before and two hours after the treatment with Fluvoxamine or PRE-084 in Timothy syndrome cardiomyocytes, although there could be a reduction in p35 protein expression with long-term (seven days) treatment of Fluvoxamine (FIGS. 4D-4F).

Furthermore, the protein expression of cdk5 before and two hours after the treatment with PRE-084, was examined. These results suggest that there was a significant reduction in cdk5 protein expression in Timothy syndrome cardiomyocytes two hours after the treatment of 5 μM PRE-084, compared with the Timothy syndrome cardiomyocytes without the treatment (FIGS. 4G-4H). This could explain the reduction in cdk5 activity after the treatment of PRE-084 in Timothy syndrome cardiomyocytes. Taken as a whole, the results suggest that one of the mechanisms underlying the beneficial effects of sigma 1 receptor agonist PRE-084 on Timothy syndrome cardiomyocytes is that PRE-084 reduces cdk5 activity by reducing cdk5 protein expression and improving the functions of Timothy syndrome cardiomyocytes, which is consistent with the finding that cdk5 inhibition alleviates many, and in some cases all of the phenotypes in Timothy syndrome cardiomyocytes in the present model system. Additionally, a direct interaction between sigma 1 receptor and cdk5 was detected in control and Timothy syndrome cells using endogenous immunoprecipitation experiments (FIG. 4I). Thus, sigma 1 receptor could also affect cdk5 directly by affecting the conformation of cdk5, leading to a change in cdk5 activity.

Finally, the effects of a combination of PRE-084 treatment and two of the positive compounds identified from the chemical testing of Roscovitine analogs and cdk5 inhibitors, were analyzed. Myoseverin-B is a Roscovitine analog that has positive effects on Timothy syndrome cardiomyocytes and PHA-793887 is a potent cdk5 inhibitor that have positive effects on Timothy syndrome cardiomyocytes. The effects of PRE-084 or Myoseverin-B or PHA-793887 alone, PRE-084 with Myoseverin-B, and PRE-084 with Myoseverin-B and PHA-793887 treatment on the abnormal contractions in Timothy syndrome cardiomyocytes were analyzed. The results suggest that the addition of Myoseverin-B or Myoseverin-B and PHA-793887 to a low dose PRE-084 treatment could enhance the spontaneous beating rate and reduce the contraction irregularity in Timothy syndrome cardiomyocytes more significantly, compared with a low dose PRE-084 treatment alone. The combination treatments also allow for the use of each compound at a lower dose to achieve the therapeutic effects (FIGS. 4J-4K). Thus, in certain embodiments, a combination of sigma 1 receptor agonists (PRE-084 or fluvoxamine) with cdk5 inhibitors and/or the Roscovitine analogs could also be a new therapeutic option for treating Timothy syndrome.

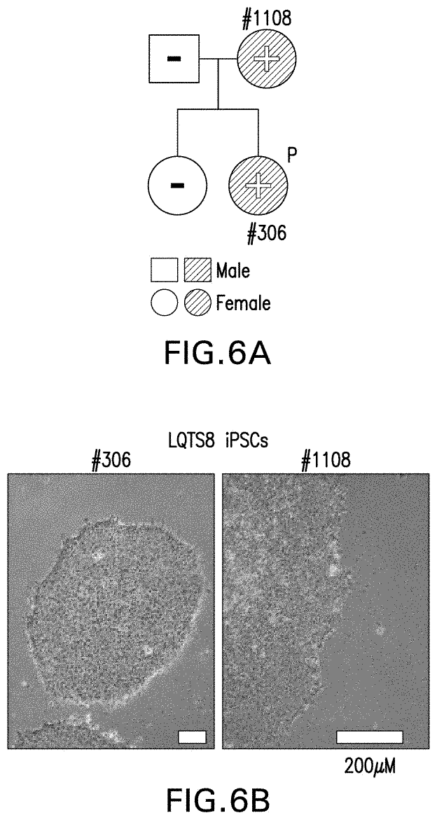

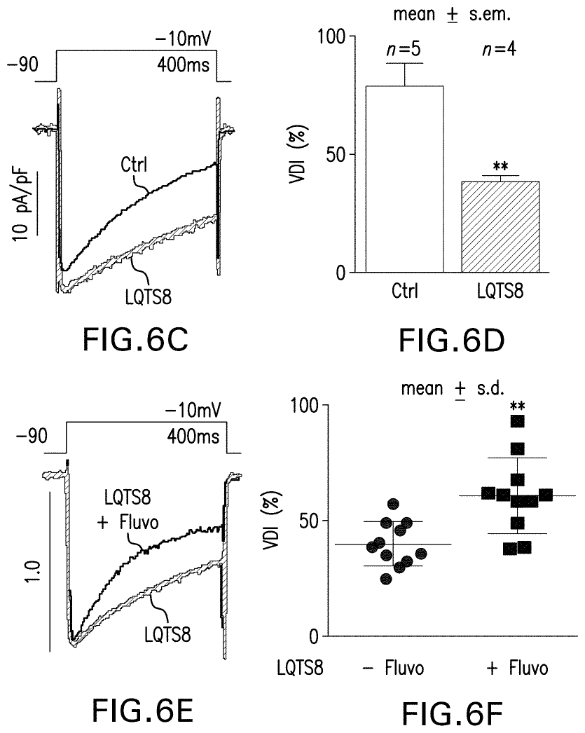

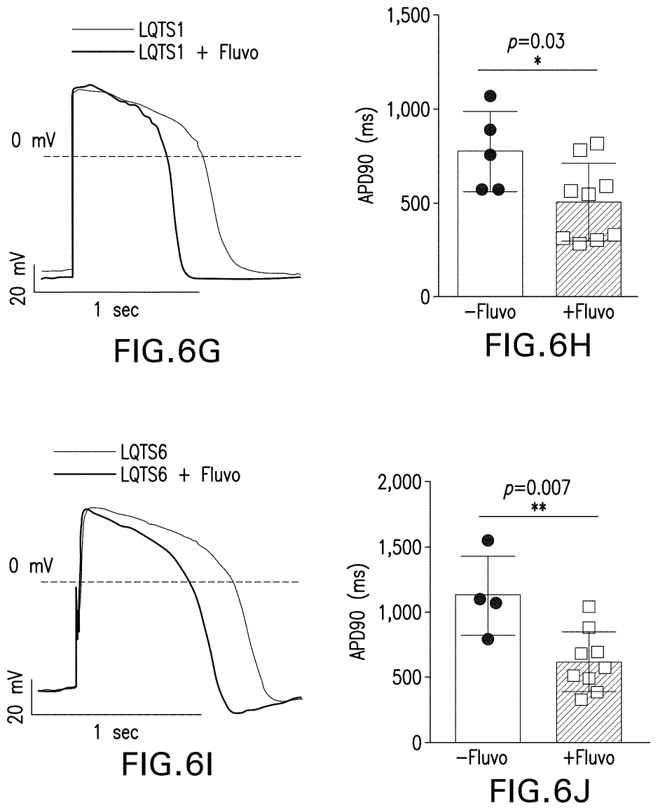

In addition to Timothy syndrome, the effects of Fluvoxamine treatment on the iPSC derived cardiomyocytes from patients with inherited LQT8, LQT1 and LQT6 were examined. As introduced previously, mutations in CACNA1C cause LQT8 and the A582D mutation in CACNA1C leads to an ineffective channel inactivation, similar to the Timothy syndrome mutation although to a less extent. A family with the A582D mutation in CACNA1C was identified and iPSCs were derived from the two patients carrying the mutation in the family (FIGS. 6A-6B). We differentiated the iPSCs into cardiomyocytes and evaluated the voltage-dependent inactivation of Cav1.2 channels in control and LQTS8 (caused by the A582D mutation) cardiomyocytes. The LQTS8 cardiomyocytes demonstrated a delayed voltage-dependent inactivation of Cav1.2 channels compared with control cardiomyocytes and the treatment of Fluvoxamine enhanced the voltage-dependent inactivation of Cav1.2 channels in LQTS8 cardiomyocytes (FIGS. 6C-6F). The results indicated a beneficial effect of Fluvoxamine treatment on LQTS8 cardiomyocytes. We also examined the effect of Fluvoxamine on the prolong paced action potentials in the cardiomyocytes derived from a LQT1 patient with the G269S mutation in KCNQ1 and a LQT6 patient with the M54T mutation in MiRP1 protein. The treatment of Fluvoxamine significantly reduced the prolonged paced action potentials in both LQTS1 cardiomyocytes and LQTS6 cardiomyocytes (FIGS. 6G-6J), indicating a beneficial effect.

In summary, these results suggest that sigma 1 receptor agonists have therapeutic effects on the cardiac phenotypes in long QT syndromes, in particular Timothy syndrome, LQT8, LQT1 and LQT6.

### Methods

iPSC maintenance.

iPSCs were cultured with Essential 8 media with 100 unit/ml penicillin and 100 μg/ml streptomycin on plates or dishes (Corning) coated with Geltrex (Life Technologies) following the manufacturer's instruction. The iPSCs were differentiated into cardiomyocytes following a monolayer based protocol that we reported previously (Song et al., 2015).

The Analysis of Cardiomyocyte Contractions for Compound Test.

The working solution of each compound was made by diluting the stock solution in our cardiomyocyte culture media to a final concentration of 5 μM or 1 μM. The contraction analysis was performed as reported previously (Yazawa et al., 2011). The movies were taken before the treatment, and 2 hours after the treatment of each compound on the intact monolayer Timothy syndrome cardiomyocytes. The contraction rate and the irregularity value of each sample before and after treatment were compared using paired Student t-test.

Patch-Clamp Electrophysiology.

The Timothy syndrome and control iPSCs were differentiated into cardiomyocytes following a protocol reported previously (Song et al., 2015). The cardiomyocytes were dissociated into single cells for whole-cell patch-clamp recordings at day 31 and used for patch clamping between day 35 and 45. Whole-cell patch-clamp recordings of iPSC-derived cardiomyocytes were conducted using a MultiClap 700B patch-clamp amplifier (Molecular Devices) and an inverted microscope equipped with differential interface optics (Nikon, Ti-U). The glass pipettes were prepared using borosilicate glass (Sutter Instrument, BF150-110-10) using a micropipette puller (Sutter Instrument, Model P-97). Voltage-clamp measurements were conducted using an extracellular solution consisting of 5 mM BaCl₂, 160 mM TEA and 10 mM HEPES (pH7.4 at 25° C.) and a pipette solution of 125 mM CsCl, 0.1 mM CaCl₂, 10 mM EGTA, 1 mM MgCl2, 4 mM MgATP and 10 mM HEPES (pH 7.4 with CsOH at 25° C.). The protocol for voltage-clamp recordings was that cells were held at −90 mV and then depolarized to −10 mV for 400 ms at a rate of 0.1 Hz for the Ba²⁺ current recordings; cells were held at −90 mV, stimulated with a 2-s family of pulses from −90 to +20 for the current-voltage relationship of the Ba²⁺ currents. The recordings were conducted under room temperature. Current-clamp recordings were conducted in normal Tyrode solution containing 140 mM NaCl, 5.4 mM KCl, 1 mM MgCl2, 10 mM glucose, 1.8 mM CaCl₂ and 10 mM HEPES (pH7.4 with NaOH at 25° C.) using the pipette solution: 120 mM K D-gluconate, 25 mM KCl, 4 mM MgATP, 2 mM NaGTP, 4 mM Na2-phospho-creatin, 10 mM EGTA, 1 mM CaCl₂ and 10 mM HEPES (pH 7.4 with KCl at 25° C.). The recordings were conducted at 37° C. Cardiac action potentials were stimulated (2 ms, 1 nA) in current clamp mode at 37° C. (0.2 or 0.5 Hz). Recorded action potentials were analyzed using Clampfit 10.4 (Axon Instruments). Control recordings were obtained from cardiomyocytes without treatment. The after treatment recordings were obtained from cardiomyocytes that had been treated with either Fluvoxamine or PRE-084 at the dose of 5 μM for two hours in the culture media.

Calcium Imaging.

For the calcium transient recordings to examine the effects Fluvoxamine or PRE084 on the abnormal spontaneous calcium transients in Timothy syndrome cardiomyocytes, the cardiomyocytes were prepared with the same experimental schedule as described in electrophysiology method section. The Nikon automatic microscope (Nikon Eclipse TiE with a motorized stage) connected to sCMOS camera (Andor Zyla sCMOS 4.2 MP) together with a stage top incubator (at 37° C., 5% CO₂ and 20% O₂, controlled by TOKAI HIT Hypoxia gas delivery system) were used for this experiment. Nikon objective lens 40× (Nikon CFI Plan Apo Lambda, NA 0.95) was used for single cell recordings and the cardiomyocyte culture media was used as bath solution. The cells were loaded with FluoForte calcium dye for calcium imaging following the manufacturer's instruction (ENZ-51016, Enzo Life Sciences). A basal recording was acquired from each cell before the treatment of Fluvoxamine or PRE-084 and after the basal recording, the solution was changed to cardiomyocyte culture media with 5 μM Fluvoxamine or PRE-084. Thirty minutes after the treatment of Fluvoxamine or PRE-084, a second recording was acquired from the same cell. The calcium transient duration and the calcium transient half (50%) decay time from the recordings before and after treatment were analyzed. Paired student's t-test was used for statistics.

In Vitro CDK5 Activity Assay.

To examine the endogenous CDK5 activity, an CDK5 activity assay was conducted following the previous reported protocols (Bu et al., 2002; Hallows et al., 2003) with some modifications. iPSC-derived cardiomyocytes differentiated from Timothy syndrome iPSCs were collected at day 26 or day 27 for the assay. For the compound-treated groups, the cardiomyocytes were treated with either Fluvoxamine or PRE-084 at the dose of 5 μM or 10 μM for two hours before being collected for the assay. Cardiomyocytes were isolated from monolayer culture and lysed with the cell lysis buffer containing 1% NP-40, 20 mM Tris-HCl, 137 mM NaCl, lx protease inhibitor cocktail, lx phosphatase inhibitor cocktail 3 and 1× phosphatase inhibitor cocktail, pH 7.4. The protein concentration in the samples was measured using a standard bicinchoninic acid (BCA) assay kit. 40 μg of proteins from each sample were aliquoted and used as one sample for CDK5 immunoprecipitation. The sample was incubated with CDK5 antibody-conjugated agarose beads (cdk5 (J-3) AC, catalog #: sc-6247 AC, Santa Cruz Biotechnology) for 2 hours at 4° C. (rocking continuously) for CDK5 immunoprecipitation. 5p1 resuspended bead solution was used for the immunoprecipitation of CDK5 from each sample. After immunoprecipitation, the beads were washed three times with cold TBS and twice with room-temperature TBS. A reaction mix containing 1× Reaction Buffer A, 50 μM DTT, 50 μM ATP, 1 μg Histone H1 in distilled water was added to each sample for detecting CDK5 activity. The stock of PHA-793887 was diluted with DMSO and added to the corresponding samples in the PHA-793887 treated TS groups at the concentration of 5 μM. The same volume of DMSO was added to the rest of the samples to achieve the same concentration of DMSO in all the reactions. A series of samples for a standard curve were prepared based on the manufacturer's instructions to determine the ATP-ADP conversion from the luminescence signals in every round of experiment. The kinase reaction tubes with the reaction mixes were incubated at 26-27° C. for 60 minutes for the kinase reaction. The ADP-Glo™ reagent was then added to the reactions for an incubation of 40 minutes at 26-27° C. to deplete the ATP in the reactions. Next the detection reagent was added to the reactions for an incubation of 45 minutes at 26-27° C. 20 μl of the sample from each tube was then transferred into a 96 well microplate and the luminescence was measured with the GloMax® 96 Microplate Luminometer (Promega) with an integration time of 1.5 s. The luminescence values were converted into the ATP-ADP conversion values based on the standard curve. Four rounds of experiment were conducted. The values from each round of experiments were normalized to the average value of the Timothy syndrome cardiomyocyte samples without PHA addition before being pooled together for the final analysis that is showed in FIG. 4A.

Western Blot and Co-Immunoprecipitation Analysis.

Anti-p35 (Rabbit polyclonal Ab, Catalog # sc-820, Clone # C-19, 1:1,000 dilution, Santa Cruz), Anti-cdk5 antibody (Mouse monoclonal Ab, Catalog # MABS50, Clone #1H3, 1:1000 dilution, millipore), Anti-sigma 1 receptor antibody (rabbit polyclonal ab, catalog # ab53852, 1:1000 dilution, abcam) and Anti-beta-Tubulin antibody (Mouse mAb, Catalog # T5201, Clone # TUB 2.1, 1:4,000 dilution, Sigma Aldrich) were used for western blotting and co-immunoprecipitation. For western blot analysis, the cardiomyocytes were used at day 26 or day 27 after differentiation. Some of the Timothy syndrome cardiomyocytes were treated with 5 μM PRE-084 or Fluvoxamine in cardiomyocyte culture media for one or two hours or seven days before being collected for western blot analysis. The Timothy syndrome cardiomyocytes from the same round of differentiation were collected as control. The cells were isolated from the monolayer and lysed with the cell lysis buffer containing 1% Triton X-100, 50 mM Tris-HCl, 150 mM NaCl, 250 mM sucrose, 1× protease inhibitor cocktail, lx phosphatase inhibitor cocktail 3 (Catalog # P0044, Sigma-Aldrich) and 1× phosphatase inhibitor cocktail (Catalog # P8340, Sigma-Aldrich), pH 7.4. The concentration of total proteins in each sample was measured using a standard bicinchoninic acid (BCA) assay kit (Pierce Biotechnology). Next, 20 μg of proteins from each sample were aliquoted and denatured with the sample buffer containing Urea and being boiled for 5 minutes at 95° C. The samples were loaded to the Tris-HCl based SDS-PAGE gels with 5% stacking gel and 10% separation gel along with the ladder. The proteins were electro-transferred to PVDF-membranes using the XCell SureLock™ Mini-Cell system overnight. Next day, the membranes were blocked with the SuperBlock Blocking for 30 minutes at RT and incubated with the primary antibody (diluted in the SuperBlock Blocking buffer) overnight at 4° C. On the third day, the membranes were incubated with the corresponding secondary antibody (1:8,000 dilution in the SuperBlock Blocking buffer) for 30 minutes at RT and then incubated with the Pierce ECL western blotting substrate followed by exposing to X-ray films in a dark room. For sequential immunoblotting, the membranes were stripped with the stripping buffer containing 62.5 mM Tris-HCl, 2% SDS, 114 mM beta-mercaptoethanol at 42° C. for 15-20 minutes, and washed six times with PBS. The stripped membranes were then re-blocked with SuperBlock Blocking Buffer for the next immunoblotting. For the immunoblotting of beta-tubulin, the membrane was stripped, re-blocked with the SuperBlock Blocking Buffer and incubated with the beta-tubulin antibody at RT for 30 minutes. The rest of the steps were the same. The western blot analysis to examine p35 protein expression in Timothy syndrome cardiomyocytes before and after 5 μM Fluvoxamine treatment was repeated eight times with independent samples from different rounds of differentiation for validation. The western blot analysis to examine p35 expression in Timothy syndrome cardiomyocytes before and after 5 μM PRE-084 treatment was repeated seven times with independent samples from different rounds of differentiation for validation. Western blot band intensities were quantified using Image J and the values were normalized to the corresponding beta-tubulin band intensities. The values from Timothy syndrome cardiomyocytes two hours after the treatment of 5 μM PRE-084 or Flvuoxamine were normalized to the values from Timothy syndrome cardiomyocytes without treatment for the presentation in the corresponding figure. For the co-immunoprecipitation, control and Timothy syndrome cardiomyocytes at day 26 or day 27 were lyzed with the cell lysis buffer containing 1% NP-40, 20 mM Tris-HCl, 137 mM NaCl, lx protease inhibitor cocktail, lx phosphatase inhibitor cocktail 3 and 1× phosphatase inhibitor cocktail, pH 7.4. The protein concentration in the samples was measured using a standard bicinchoninic acid (BCA) assay kit. 40 μg of proteins from each sample were aliquoted and used as one sample for CDK5 immunoprecipitation. The sample was incubated with CDK5 antibody-conjugated agarose beads (cdk5 (J-3) AC, catalog #: sc-6247 AC, Santa Cruz Biotechnology) for 2 hours at 4° C. (rocking continuously) for CDK5 immunoprecipitation. 5₁11 resuspended bead solution was used for the immunoprecipitation of CDK5 from each sample. After immunoprecipitation, the beads were washed five times with cold TBS. The sample buffer containing Urea (2× stock, 8M Urea, 40 mM Tris-HCl, 2% sodium dodecyl sulfate (SDS), 10% beta-mercaptoethanol and 0.01% bromophenol blue) was added to the beads and the samples were boiled for 5 minutes at 95° C. for denaturing the proteins. The samples were then loaded to Tris-HCl based SDS-PAGE gels with 5% stacking gel and 10% or 15% separation gel along with the ladder (Prestained SDS-PAGE standards, broad range, catalog #161-0318, Biorad). The proteins were electro-transferred to PVDF-membranes (Invitrolon™ PVDF, Catalog # LC2005, NOVEX by Life Technologies) using the XCell SureLock™ Mini-Cell system (Invitrogen) overnight at 4° C. Next day, the membranes were blocked with the SuperBlock Blocking Buffer in PBS (phosphate-buffered saline, Catalog #27515, Thermo Fisher Scientific) for 30 minutes at room temperature (RT) and then incubated with the primary antibody (diluted in the SuperBlock Blocking buffer) overnight, followed by an incubation of the corresponding secondary antibody (Thermo Fisher Scientific, Pierce, anti-mouse #31430; anti-rabbit, #31460, 1:8,000 dilution in the SuperBlock Blocking buffer) for 30 minutes at RT. The membranes were then incubated with the Pierce ECL western blotting substrate (Catalog #32209, Thermo Fisher Scientific) followed by exposing to X-ray films (CL-X Posure™ film, Catalog #34091, Thermo Fisher Scientific) in a dark room. The co-IP experiments were repeated three times to confirm the results and the representative image was shown.

Quantitative RT-PCR.

RNA from the Timothy syndrome cardiomyocytes before and one or two hours after the treatment of 5 μM PRE-084 or 5 μM Fluvoxamine was prepared using the RNeasy Mini kit and RNase-Free DNase set (Qiagen). cDNA was synthesized using the SuperScript III First-Strand Synthesis System for RT-PCR (Life Technologies). The cDNA (21 μl) was diluted with DNase-free water (Invitrogen) at 1:1 and 1 μl of the samples was used for qPCR analysis. SYBR Advantage qPCR Premix (Clontech/TaKaRa Bio) and StepOnePlus real time PCR systems (Life Technologies) were used for qPCR. The primer sets for detecting the CDK5 and GAPDH transcripts were as follows: CDK5 Forward 5′-GGCTTCAGGTCCCTGTGTAG-3′ (SEQ ID NO:1), Reverse 5′-ATGGTGACCTCGATCCTGAG-3′ (SEQ ID NO:2); GAPDH, Forward 5′-GATGACATCAAGAAGGTGGTGA-3′ (SEQ ID NO:3), Reverse 5′-GTCTACATGGCAACTGTGAGGA-3′ (SEQ ID NO:4). The CT value of each sample at 50% of the amplification curve was used and GAPDH was used to normalize the expression of CDK5.

Statistical Analysis.

The statistics used for every figure have been indicated in the corresponding figure legends. The Student t-test (paired and unpaired) was conducted with the t-test functions in Microsoft Excel software. The Student t-test was two tails. The One-way ANOVA with Bonferroni or Dunnett's posthoc analysis was conducted with the Graphpad prism software. All the data meet the assumptions of the statistical tests. All the samples used in this study were biological repeats, not technical repeats. No samples were excluded from the analysis in this study.

### Example 2—Dextromethorphan for Treating LQTS Including Timothy Syndrome

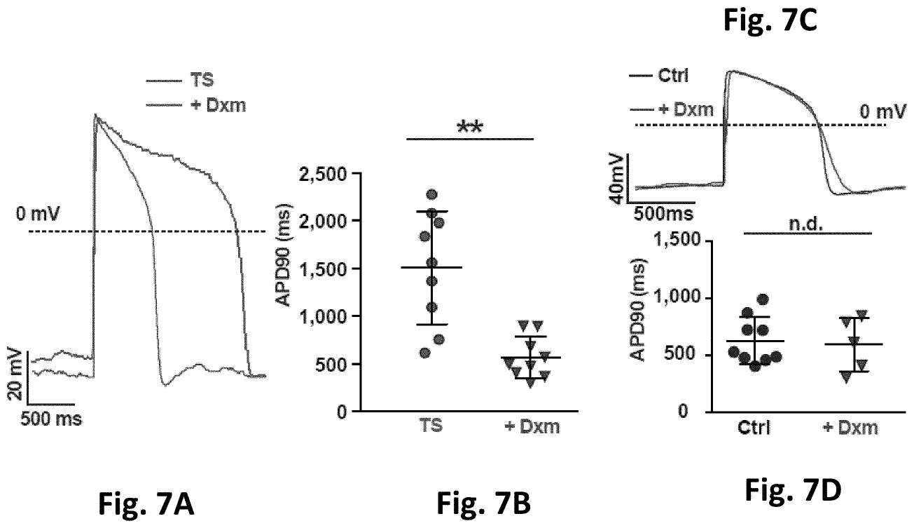

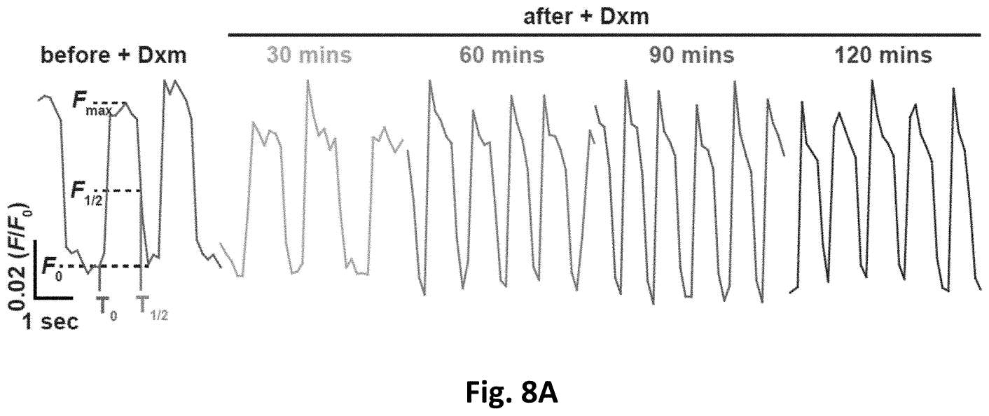

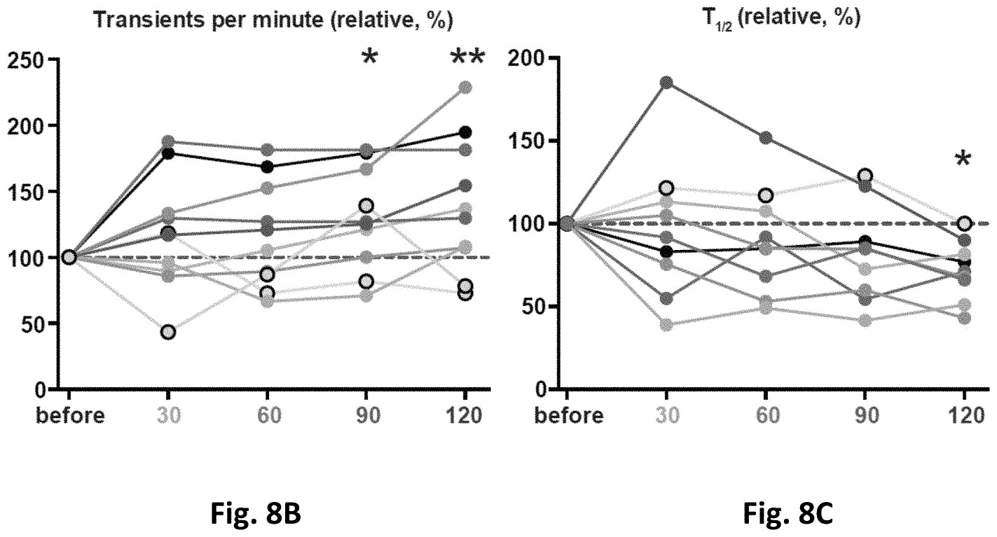

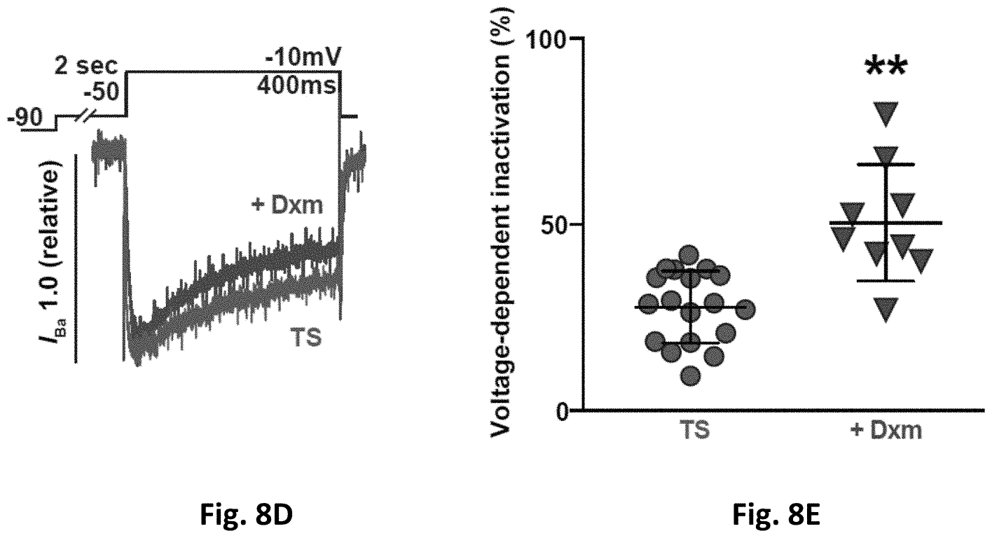

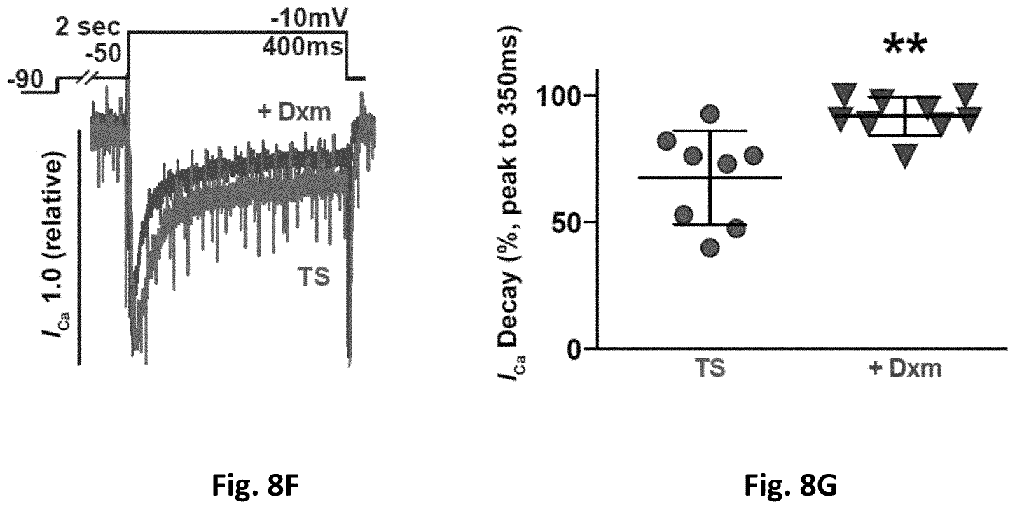

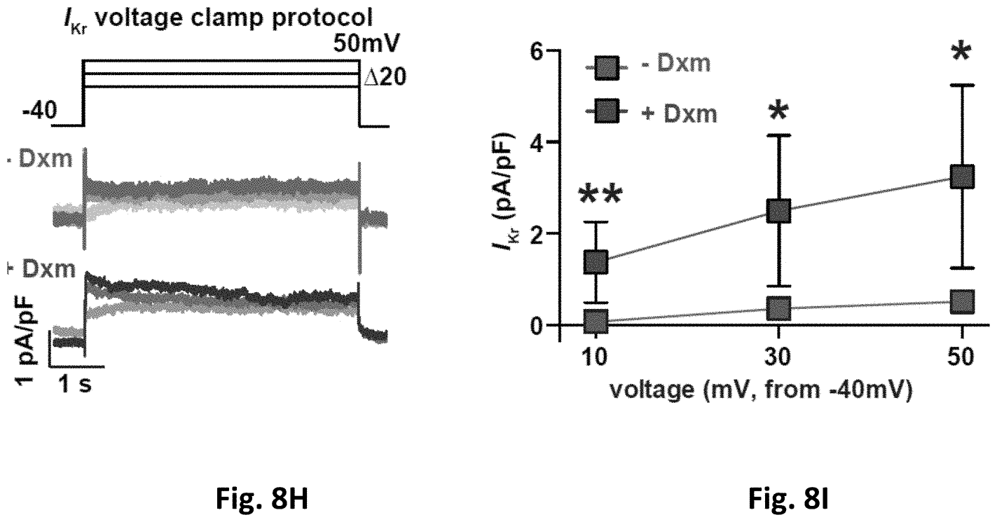

The results herein show that dextromethorphan (Dxm), an FDA-approved cough suppressant, restored cardiac action potential (FIGS. 7A and 7B), calcium handling and ion channel function in human Timothy syndrome iPSC-derived cardiomyocytes (FIGS. 8A-8G). On the other hand, action potential duration was not changed by Dxm in the isogenic control cells (FIGS. 7C and 7D). This result suggested that Dxm does not have any significant adverse effect on the electrophysiological properties of healthy control cardiomyocytes, which is consistent with the fact that there are no clinical reports regarding significant adverse cardiovascular effects of Dxm on human subjects.

Dxm is also known as a Sigma-1R agonist (Ki, 150 nM) as PRE-84 (Ki, 2.2 nM) and fluvoxamine (Ki, 36 nM). Therefore, Dxm may alleviate the phenotypes in Timothy syndrome cardiomyocytes in the same molecular mechanism as fluvoxamine and PRE-084, which is through activating Sigma-1R in Timothy syndrome cardiomyocytes.

Interestingly, it was also found that Dxm significantly increased hERG current I_{Kr} in Timothy syndrome iPSC-derived cardiomyocytes (FIGS. 8H and 8I). This result suggested that increased calcium channel inactivation, reduced calcium transients and increased hERG currents have contributed to shortened action potentials in Timothy syndrome iPSC-derived cardiomyocytes.

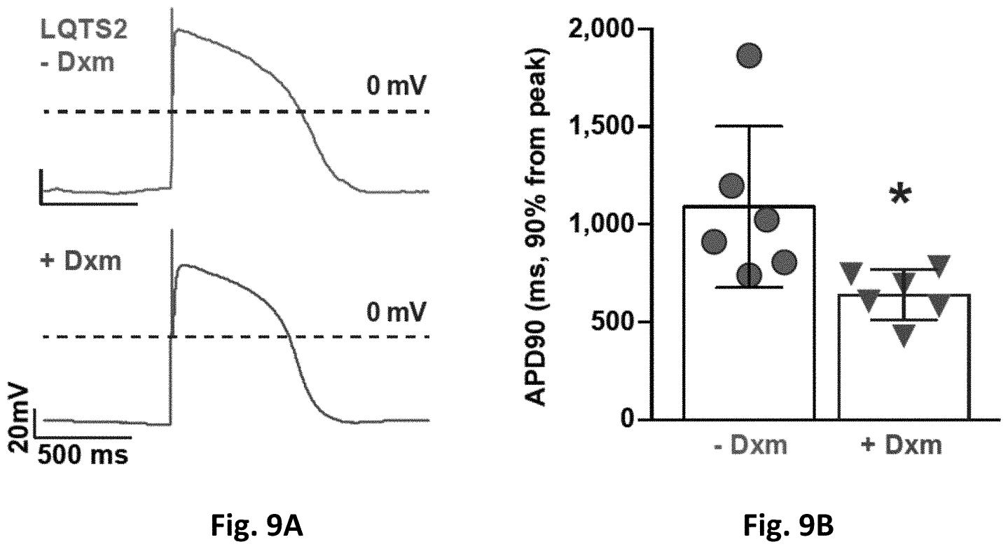

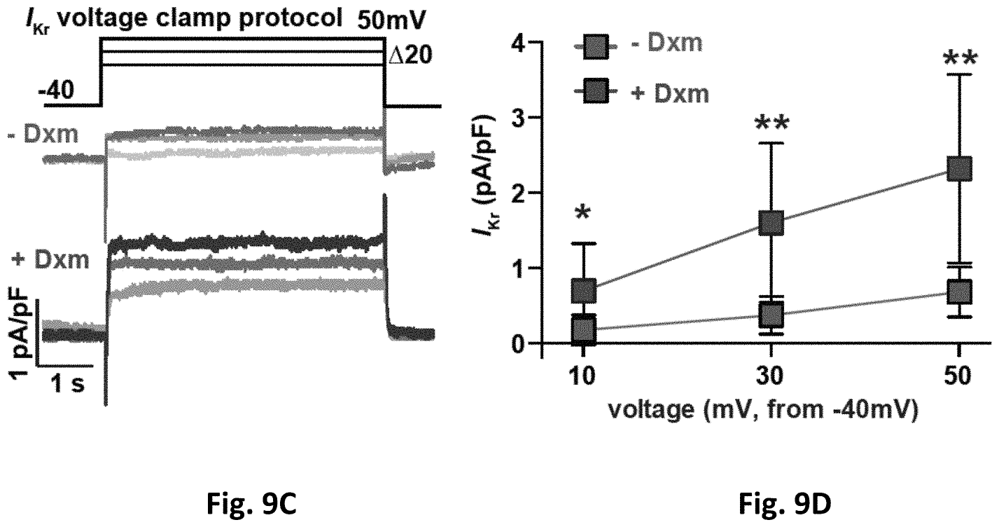

Based on the positive effect of Dxm on hERG current in Timothy syndrome iPSC-derived cardiomyocytes, it was hypothesized that Dxm treatment could be beneficial for long QT syndrome type 2 (LQTS2), which is a major form of long QT syndrome caused by hERG channel missense mutations. Consistent with this hypothesis, it was found that Dxm significantly shortened action potentials and also increased hERG current I_{Kr} in LQTS2 iPSC-derived cardiomyocytes (FIGS. 9A-9D).

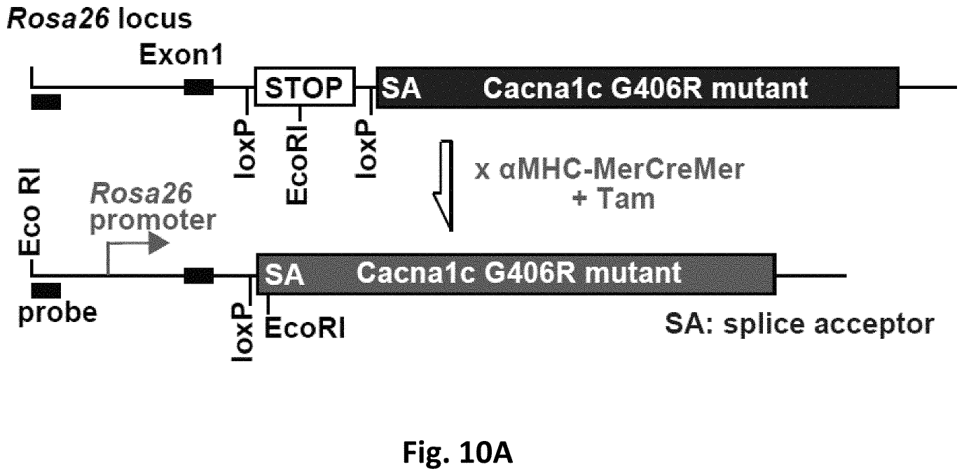

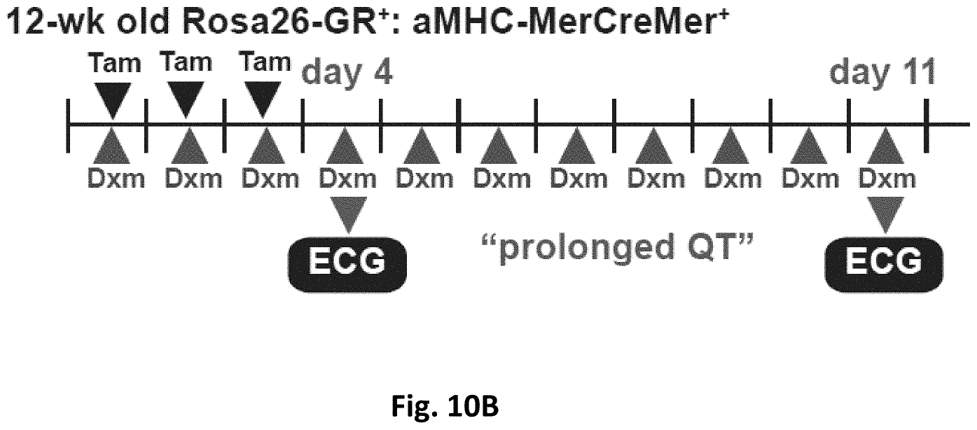

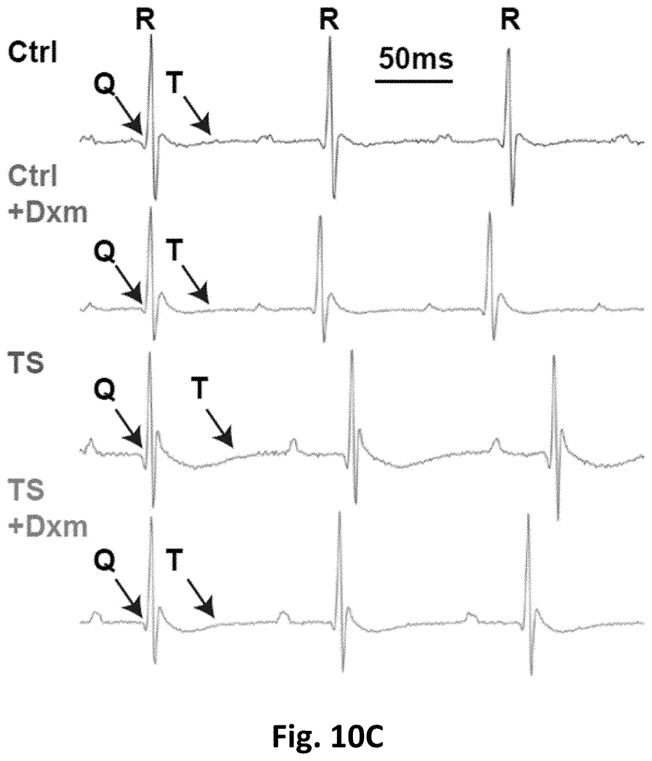

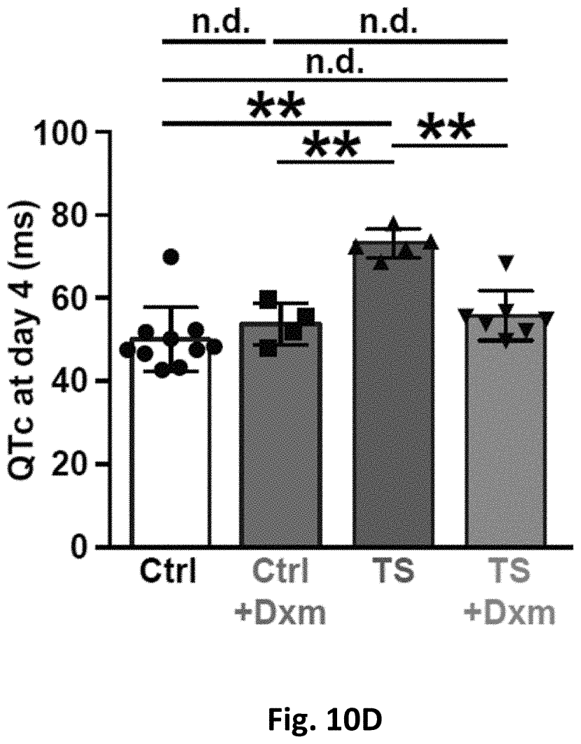

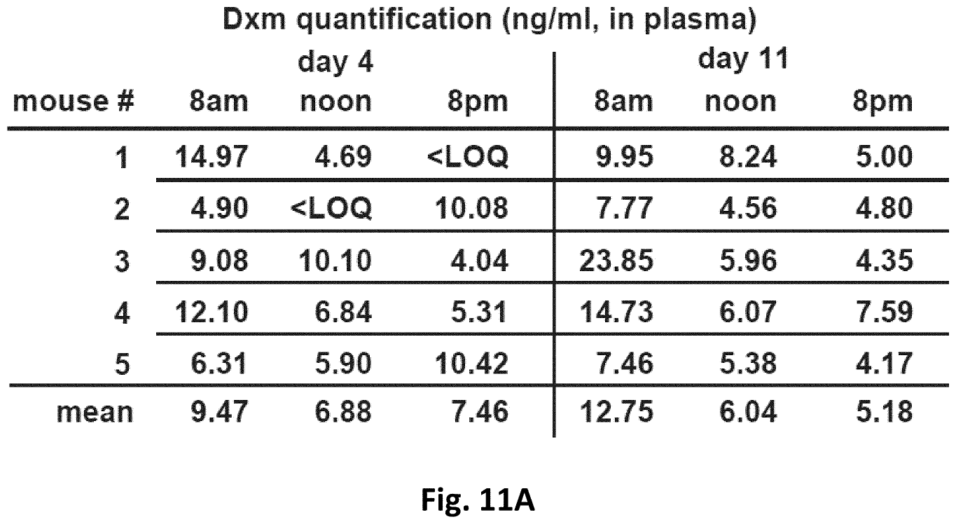

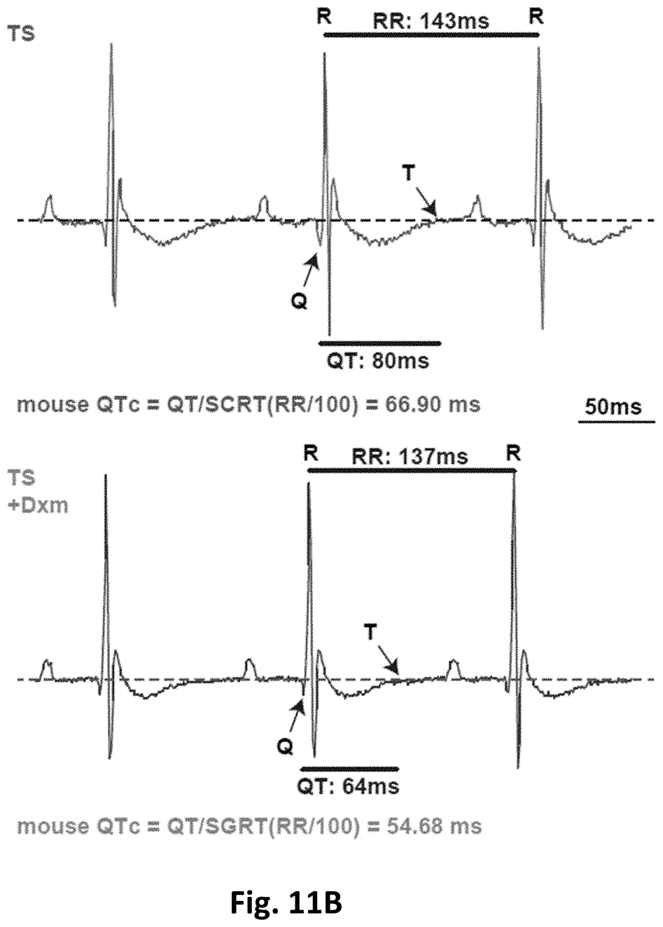

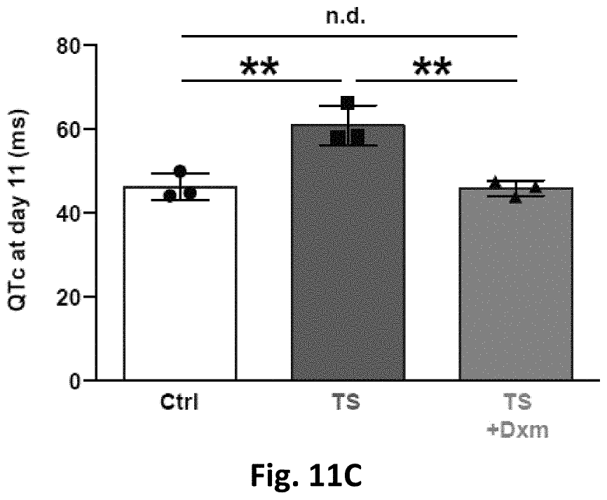

Next, in order to investigate whether the in vitro findings could be translated into in vivo applications, the effect of Dxm on the cardiac phenotypes in Timothy syndrome mouse model was examined. Timothy syndrome mice demonstrated a significantly prolonged QT intervals in ECG compared with control littermates and Dxm significantly shortened the QT intervals in Timothy syndrome mice while there was no significant effect of Dxm observed on the QT intervals in the control littermate mice (FIGS. 10A-10D and FIGS. 11A-11D). Dxm concentration in the plasma of adult mice treated with Dxm (in drinking water) were examined at different time points (FIG. 11A) and the results confirmed the proper exposure of Dxm in the animals during the studies with this dosing method. In addition, the plasma concentration of Dxm in the study (5-10 ng/ml) was also within the normal dose range of Dxm used in clinical practice.

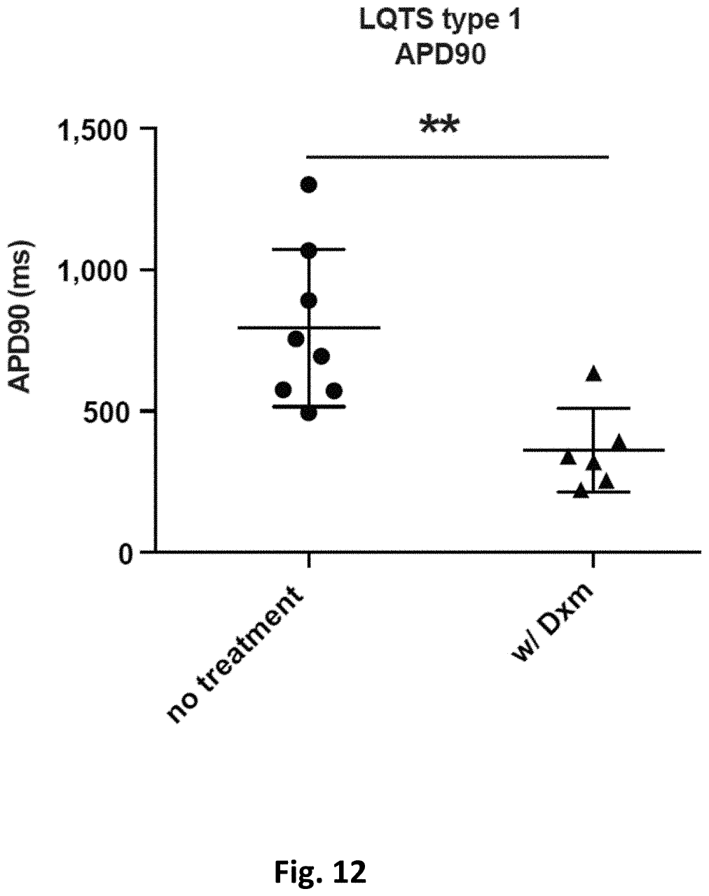

At least, it was also found that Dxm significantly shortened action potentials in LQTS1 iPSC-derived cardiomyocytes (FIG. 12). The result suggested that Dxm is beneficial not only for Timothy syndrome, a rare form of long QT syndrome but also for the major forms LQTS 1 and 2.

### Methods

Unless otherwise noted below, the same methods used in Example 1 were used in Example 2.

Ipsc Cells.

iPSCs were cultured with Essential 8 media with 100 unit/ml penicillin and 100 μg/ml streptomycin on plates or dishes (Corning) coated with Geltrex (Life Technologies) following the manufacturer's instruction. The iPSCs were differentiated into cardiomyocytes following a monolayer based protocol that we reported previously (Song et al., 2015). The isogenic control lines have been established using gene editing technology TALEN for correcting Timothy syndrome mutation G406R in human CACNA1C gene.

Dextromethorphan Treatment.

The working solution of each compound was made by diluting the stock solution in the cardiomyocyte culture media to a final concentration of 5 μM.

Patch-Clamp Electrophysiology of the cardiomyocytes was done as described in Example 1.

GCaMP6f-Based Calcium Imaging.

Human iPSC-derived cultured cardiomyocytes were dissociated to isolate cardiomyocyte cell clusters and plated onto glass-bottom dishes Instead of FluoForte calcium dye loading described in Example 1, during the dissociation process, the cells were infected with GCaMP6f adeno associate virus (AAV-GCaMP6f, University of Pennsylvania Vector Core). The isolated cardiomyocytes were then imaged interrogating their YFP fluorescence signal (508 nm) using a Nikon automatic microscope. As described in Example 1, each experiment was conducted using Timothy syndrome cardiomyocytes. The baseline measurements were taken for 30 seconds for each cell after which the bath solution was replaced with DF5 supplemented 5 uM Dxm (Sigma-Aldrich). Upon replacement of the media with DF5+Dxm, the cardiomyocytes were allowed to equilibrate for 30 minutes and were then imaged again. The calcium transient half (50%) decay time as well as amplitude for each cell were then quantified. The data for each cell was normalized to the initial baseline reading as a percent change.

I_{Kr} Current Recording.

Voltage-clamp recording for I_{Kr} measurements was conducted in normal Tyrode's solution containing 140 mM NaCl, 5.4 mM KCl, 1 mM MgCl2, 10 mM glucose, 1.8 mM CaCl₂) and 10 mM HEPES (pH 7.4 with NaOH at 25° C.) supplemented with 10 μM nisoldipine (I_{Ca} current blocker, Sigma-Aldrich), 10 μM Chromanol-293B (I_{Ks} current blocker, TOCRIS) using the pipette solution 120 mM K d-gluconate, 25 mM KCl, 4 mM MgATP, 2 mM NaGTP, 4 mM Na2-phospho-creatine, 10 mM EGTA, 1 mM CaCl₂) and 10 mM HEPES (pH 7.4 with KCl at 25° C.). The following pulse protocol was used: 5-s voltage clamp applied from 10 mV to 50 mV (Δ20 mV, holding at −40 mV, 0.1 Hz, at 25° C.). After recordings, the extracellular solution was switched to one containing 1 μM E-4031 (TOCRIS). E-4031-sensitive current was measured and analyzed as I_{Kr} current using Clampfit 10.4 (Axon Instruments).

Timothy Syndrome Mouse Model.

A model mouse for Timothy Syndrome was generated by targeting the Rosa26 locus with a G406R mutant in the calcium channel Cacna1c gene (see FIG. 10A). The experimental design for inducing the expression of Timothy syndrome mutant with Dxm treatment (about 28 mg/kg) is shown in FIG. 10B.

Mouse Electrocardiography.

Subcutaneous 4-lead electrocardiograms of isoflurane-anesthetized 12-week old mice were performed using emka ECG and recorded using iox 2.8.0.19. Recordings were conducted a day 4 and 11 after tamoxifen (Sigma-Aldrich, 50 μg/B.W.(g)/day/mouse, i.e., approximately 1 mg/day/mouse) was administrated at day 1-3. Dxm was resolved to drinking water. Total amount of taken dextromethorphan was estimated based on water consumption in each mouse (23-44 mg/kg). To obtain accurate amount of dextromethorphan administration in mice, the concentration of dextromethorphan in the plasma was measured and analyzed at Columbia University Biomarkers Core Laboratory. RR and QT intervals were measured manually using ecgAUTO v2.8.1.27, Excel and Prism 6/7 software.

### REFERENCES

Huikuri H V, et al. (2001). Sudden death due to cardiac arrhythmias. N Engl J Med. 345(20):1473-82.
Crotti L, et al. (2013). Calmodulin mutations associated with recurrent cardiac arrest in infants. Circulation. 127(9): 1009-17.
Mahida S, et al. (2013). Genetics of congenital and drug-induced long QT syndromes: current evidence and future research perspectives. Journal of interventional cardiac electrophysiology: an international journal of arrhythmias and pacing. 37(1):9-19.
Venetucci L, et al. (2012). Inherited calcium channelopathies in the pathophysiology of arrhythmias. Nature reviews Cardiology. 9(10):561-75.
Paakkari I. (2002). Cardiotoxicity of new antihistamines and cisapride. Toxicol Lett. 127(1-3): 279-84.
Splawski I, et al. (2004). Ca(V)1.2 calcium channel dysfunction causes a multisystem disorder including arrhythmia and autism. Cell. 119(1): 19-31.
Balasuriya, D., Stewart, A. P., Crottes, D., Borgese, F., Soriani, O., and Edwardson, J. M. (2012). The sigma-1 receptor binds to the Nav1.5 voltage-gated Na+ channel with 4-fold symmetry. J Biol Chem 287, 37021-37029.
Bu, B., Li, J., Davies, P., and Vincent, I. (2002). Deregulation of cdk5, hyperphosphorylation, and cytoskeletal pathology in the Niemann-Pick type C murine model. J Neurosci 22, 6515-6525.
Fukuyama M, Wang Q, Kato K, et al. Long QT syndrome type 8: novel CACNA1C mutations causing QT prolongation and variant phenotypes. Europace. 2014; 16(12):1828-37.
Hallows, J. L., Chen, K., DePinho, R. A., and Vincent, I. (2003). Decreased cyclin-dependent kinase 5 (cdk5) activity is accompanied by redistribution of cdk5 and cytoskeletal proteins and increased cytoskeletal protein phosphorylation in p35 null mice. J Neurosci 23, 10633-10644.
Gao, X. F., Yao, J. J., He, Y. L., Hu, C., and Mei, Y. A. (2012). Sigma-1 receptor agonists directly inhibit Nav1.2/1.4 channels. PLoS One 7, e49384.
Giudicessi J R, Ackerman M J. Prevalence and potential genetic determinants of sensorineural deafness in KCNQ1 homozygosity and compound heterozygosity. Circ Cardiovasc Genet. 2013; 6(2): 193-200.
Lu Y, Mahaut-smith M P, Huang C L, Vandenberg J I. Mutant MiRP1 subunits modulate HERG K+ channel gating: a mechanism for pro-arrhythmia in long QT syndrome type 6. J Physiol (Lond). 2003; 551(Pt 1): 253-62.
Johannessen, M., Ramachandran, S., Riemer, L., Ramos-Serrano, A., Ruoho, A. E., and Jackson, M. B. (2009). Voltage-gated sodium channel modulation by sigma-receptors in cardiac myocytes and heterologous systems. Am J Physiol Cell Physiol 296, C1049-1057.
Tsai, S. Y., Pokrass, M. J., Klauer, N. R., Nohara, H., and Su, T. P. (2015). Sigma-1 receptor regulates Tau phosphorylation and axon extension by shaping p35 turnover via myristic acid. Proc Natl Acad Sci USA 112, 6742-6747.
Song, L., Awari, D. W., Han, E. Y., Uche-Anya, E., Park, S. H., Yabe, Y. A., Chung, W. K., and Yazawa, M. (2015). Dual optical recordings for action potentials and calcium handling in induced pluripotent stem cell models of cardiac arrhythmias using genetically encoded fluorescent indicators. Stem Cells Transl Med 4, 468-475.
Nawathe P A, Kryukova Y, Oren R V, et al. An LQTS6 MiRP1 mutation suppresses pacemaker current and is associated with sinus bradycardia. J Cardiovasc Electrophysiol. 2013; 24(9):1021-7 Wu J, Ding W G, Horie M. Molecular pathogenesis of long QT syndrome type 1. J Arrhythm. 2016; 32(5):381-388.
Yazawa, M., Hsueh, B., Jia, X., Pasca, A. M., Bernstein, J. A., Hallmayer, J., and Dolmetsch, R. E. (2011). Using induced pluripotent stem cells to investigate cardiac phenotypes in Timothy syndrome. Nature 471, 230-234.

### INCORPORATION BY REFERENCE

All references cited herein are incorporated by reference to the same extent as if each individual publication, database entry (e.g. Genbank sequences or GenelD entries), patent application, or patent, was specifically and individually indicated to be incorporated by reference. This statement of incorporation by reference is intended by Applicants, pursuant to 37 C.F.R. § 1.57(b)(1), to relate to each and every individual publication, database entry (e.g. Genbank sequences or GeneID entries), patent application, or patent, each of which is clearly identified in compliance with 37 C.F.R. § 1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

The foregoing written specification is considered to be sufficient to enable one skilled in the art to practice the invention. Various modifications of the invention in addition to those shown and described herein will become apparent to those skilled in the art from the foregoing description and fall within the scope of the appended claims. The entire disclosure of each of the patent documents, including certificates of correction, patent application documents, scientific articles, governmental reports, websites, and other references referred to herein is incorporated by reference herein in its entirety for all purposes. In case of a conflict in terminology, the present specification controls.

### EQUIVALENTS

The invention can be embodied in other specific forms without departing from the spirit or essential characteristics thereof. The foregoing embodiments are to be considered in all respects illustrative rather than limiting on the invention described herein. In the various embodiments of the methods and systems of the present invention, where the term comprises is used with respect to the recited steps or components, it is also contemplated that the methods and systems consist essentially of, or consist of, the recited steps or components. Further, it should be understood that the order of steps or order for performing certain actions is immaterial so long as the invention remains operable. Moreover, two or more steps or actions can be conducted simultaneously.

In the specification, the singular forms also include the plural forms, unless the context clearly dictates otherwise. Unless defined otherwise, all technical and scientific terms used herein have the same meaning as commonly understood by one of ordinary skill in the art to which this invention belongs. In the case of conflict, the present specification will control.

All percentages and ratios used herein, unless otherwise indicated, are by weight.

1. A method for increasing sigma-1 receptor activity in a subject in need thereof, comprising administering to the subject an effective amount of fluvoxamine or PRE-084 or dextromethorphan, or derivatives thereof, combinations thereof, or a pharmaceutically acceptable salt thereof.

2. The method of claim 1, wherein the subject exhibits one or more symptoms associated with Timothy Syndrome (TS), LQT8, LQT1, LQT2, LQT6 or a related channelopathy.

3. The method of claim 2, wherein one or more symptoms exhibit improvement and comprise any one or combination of improvements selected from the group consisting of increasing the spontaneous beating rate, decreasing the contraction irregularity, enhancing the voltage-dependent inactivation of Cav1.2 channels, rescuing the abnormal spontaneous or paced action potentials; and alleviating the abnormal spontaneous calcium transients in affected or diseased cardiomyocytes.

4. The method of claim 1, further comprising administering an effective amount of Myoseverin-B and/or PHA-793887 and/or Roscovitine and/or CR8 and/or DRF053.

5. A method for treating Timothy Syndrome (TS), LQT8, LQT1, LQT2, LQT6 or related channelopathy in a subject in need thereof comprising increasing or agonizing sigma-1 receptor activity in the subject in an amount to alleviate at least one symptom associated with TS, LQT8, LQT1, LQT2, LQT6 or related channelopathy.

6. The method of claim 5, wherein increasing the activity is by gene therapy.

7. The method of claim 5, wherein the increasing or agonizing is by administering an effective amount of fluvoxamine or PRE-084 or dextromethorphan, derivatives thereof, combinations thereof, or a pharmaceutically acceptable salt thereof.

8. The method of claim 5, wherein one or more symptoms exhibiting improvement comprise any one or combination of improvements selected from the group consisting of increasing the spontaneous beating rate, decreasing the contraction irregularity, enhancing the voltage-dependent inactivation of Cav1.2 channels, rescuing the abnormal spontaneous or paced action potentials; and alleviating the abnormal spontaneous calcium transients in affected or diseased cardiomyocytes.

9. The method of claim 7, further comprising administering an effective amount of Myoseverin-B and/or PHA-793887 and/or Roscovitine and/or CR8 and/or DRF053.

10. A method for treating or reducing risk of a cardiac arrhythmia in a subject in need thereof, comprising administering to the subject a therapeutically effective amount of fluvoxamine or PRE-084 or dextromethorphan, or derivatives thereof, combinations thereof, or a pharmaceutically acceptable salt thereof.

11. The method of claim 10, wherein one or more symptoms in the subject exhibit improvement and comprise any one or combination of improvements selected from the group consisting of increasing the spontaneous beating rate, decreasing the contraction irregularity, enhancing the voltage-dependent inactivation of Cav1.2 channels, rescuing the abnormal spontaneous or paced action potentials; and alleviating the abnormal spontaneous calcium transients in affected or diseased cardiomyocytes.

12. The method of claim 10, further comprising administering an effective amount of Myoseverin-B and/or PHA-793887 and/or Roscovitine and/or CR8 and/or DRF053.

13. The method of claim 1, wherein the subject is a mammal.

14. The method of claim 13, wherein the mammal is a human.

15. The method of claim 5, wherein the subject is a mammal.

16. The method of claim 15, wherein the mammal is a human.

17. The method of claim 10, wherein the subject is a mammal.

18. The method of claim 17, wherein the mammal is a human.


SEQUENCE LISTING (4 sequences)

SEQ ID NO: 1 - DNA, 20 bp, Artificial Sequence
  CDK5 Forward
ggcttcaggtccctgtgtag

SEQ ID NO: 2 - DNA, 20 bp, Artificial Sequence
  CDK5 Reverse
atggtgacctcgatcctgag

SEQ ID NO: 3 - DNA, 22 bp, Artificial Sequence
  GAPDH, Forward
gatgacatcaagaaggtggtga

SEQ ID NO: 4 - DNA, 22 bp, Artificial Sequence
  GAPDH Reverse
gtctacatggcaactgtgagga